(19) United States
(12) Patent Application Publication
(10) Pub. No.: US 2008/0132458 A1
(43) Pub. Date: Jun. 5, 2008

(54) Hypoxia-Activated Anti-Cancer Agents

(75) Inventors: Mark Matteucci, Portola Valley, CA (US); Jian-Xin Duan, South San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Threshold Pharmaceuticals, Inc., Redwood City, CA (US)

(21) Appl. No.: 10/592,434

(22) PCT Filed: Mar. 10, 2005
(86) PCT No.: PCT/US05/08161

§ 371 Date: Feb. 13, 2008

Related U.S. Application Data
(60) Provisional application No. 60/552,315, filed on Mar. 10, 2004.
(60) Provisional application No. 60/582,471, filed on Jun. 23, 2004.

(51) Int. Cl. A61K 31/70; A61P 35/00; C07H 15/24

(52) U.S. Cl. 514/34; 536/6.4

(57) ABSTRACT

Prodrugs of cyclic anthracyclin toxins comprising a hypoxia-activated trigger and are disclosed. In addition, methods of treating cancer using the compounds of the invention are disclosed.

### CROSS-REFERENCE TO RELATED APPLICATIONS

This application claims benefit of commonly-owned, co-pending U.S. patent application Nos. 60/552,315, filed 10 Mar. 2004; and 60/582,471 filed 23 Jun. 2004, each of which is incorporated herein by reference.

### FIELD OF THE INVENTION

The present invention provides methods, compounds, and compositions useful in the treatment of cancer and relates to the fields of chemistry, medicinal chemistry, pharmacology, and medicine.

### BACKGROUND OF THE INVENTION

The term “cancer” generally refers to one of a group of more than 100 diseases caused by the uncontrolled growth and spread of abnormal cells that can take the form of solid tumors, lymphomas, and non-solid cancers such as leukemia. Unlike normal cells, which reproduce until maturation is attained and then only as necessary for replacement, cancer cells grow and divide endlessly, crowding out nearby cells and eventually spreading to other parts of the body, unless their progression is stopped. Once cancer cells metastasize by leaving a tumor, they will travel through the bloodstream or lymphatic system to other parts of the body, where the cells begin multiplying and developing into new tumors. This sort of tumor progression makes cancer dangerously fatal. Although there have been great improvements in diagnosis, general patient care, surgical techniques, and local and systemic adjuvant therapies, most deaths from cancer are still due to metastases and other cancers that are resistant to conventional therapies including radiation and chemotherapy.

Radiation therapy is typically only effective for cancer treatment at early and middle stages of cancer, when cancer is localized, and not effective for late stage disease with metastasis. Chemotherapy can be effective at all stages of the disease, but there can be severe side effects, e.g. vomiting, low white blood cell count, loss of hair, loss of weight and other toxic effects, to both chemotherapy and radiation therapy. Because of such severe side effects, many cancer patients do not or cannot successfully complete a chemotherapy treatment regimen. The side effects of radiation and anticancer drugs can be viewed as resulting from poor target specificity. Anticancer drugs, typically administered intravenously or more rarely orally, circulate through most normal tissues of patients as well as the target tumors. If the drug is toxic to a normal cell, then this circulation will result in the death of normal cells, leading to side effects, and the more toxic the drug to normal cells, the more serious the side effects. Due to these and other problems, some highly cytotoxic chemotherapeutic agents, agents with nanomolar or sub-nanomolar IC₅₀ values against cancer cells, have not been successfully developed into approved drugs.

For example, researchers have been investigating highly potent analogs of the anthracyclines for many years. The early history of the well known cancer drug adriamycin (doxorubicin) and the numerous analogs that have been made and tested is described in the reference Henry, 1976, Cancer Chemotherapy, ACS Symposium Series, p. 15-57. Daunarubicin, another anthracycline, is also a well-known cancer drug that is structurally very similar to doxorubicin but less potent. Beginning in about 1980, researchers at SRI began making very potent analogs of daunarubicin, including 3′-deamino-3-(4-morpholinyl)daunarubicin and 3-cyanomorpholinodoxorubicin. See U.S. Pat. Nos. 4,301,277 and 4,314,054; see also U.S. Pat. Nos. 4,464,529; 4,585,859; 4,591,637; and 4,826,964; each of which is incorporated herein by reference. Such compounds were many-fold more active than daunarubicin or doxorubicin, and it was later shown that the ability of these compounds to form an aminal adduct with an amino group of a guanine base in close vicinity to the binding site of the compound resulted in the increased potency. See Nagy et al., March 1996, Proc. Natl. Acad. Sci. USA 93: 2464-2469, incorporated herein by reference. Conjugates of certain of these compounds linked to a peptide hormone tumor-targeting agent such as LH-RH. See U.S. Pat. Nos. 5,843,903 and 6,184,374, each of which is incorporated herein by reference. See also U.S. Pat. No. 5,962,216 and PCT publication No. WO 98/13059.

In about 1990, Dr. David Farquhar at the University of Texas began exploring a series of similarly highly potent doxorubicin analogs synthesized as prodrugs in which the 3′-amino group of the anthracycline was substituted with a latent alkanal or heteroalkanal group. The alkanal group converts to the corresponding free aldehyde in a biological medium, allowing the aldehyde to react with nucleophiles proximate to the DNA drug-binding site to form covalent adducts. See U.S. Pat. Nos. 5,196,522; 6,218,519; and 6,433,150 and Bakina and Farquhar, 1999, Anti-Cancer Drug Design 14: 507-515, each of which is incorporated herein by reference. Dr. Farquhar's compounds contain the basic amino group on the sugar of doxorubicin or daunorubicin, which is very important for the activity of the compounds; consequently, although masked, the prodrug compounds of Dr. Farquhar still have some activity even prior to activation. In addition, for those compounds in which the latent aldehyde is masked as a sugar acetal and released by a glycosidase, the acetal connection is chiral. It would be beneficial if highly potent anthracyclines with fewer chiral centers were available for use in cancer therapy.

Another class of “super-toxic” anthracyclines is the baminomycins. Baminomycins are a class of daunarubicin derivatives, the first members of which were isolated from microorganisms. The general structure of a compound in this class is shown below, with the R₁ and R₂ groups being alkyl with variable heteroatom substitutions. Cross-linking properties and background information on baminomycin can be found in the reference Perrin et al., 1999, Nucleic Acids Research, p. 1781.

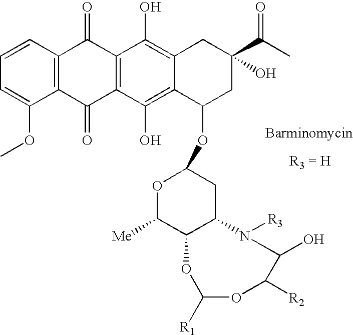

The baminomycins have not been developed as anti-cancer agents for several reasons. Although these compounds can be produced from natural organisms, this process is difficult, Further, the presence of the two chiral carbons, bearing R₁ and R₂ in the structure above, making chemical synthesis of a specific isomer very difficult and in any event expensive. See also U.S. Pat. Nos. 6,437,105 and 6,680,300, each of which is incorporated herein by reference. Production of baminomycins by fermentation may always be problematic, because microorganisms will have some sensitivity to the toxin, given that it damages DNA, even if they have a good export pump, likely limiting the yields that can be obtained. There would be significant advantages to development of a baminomycin that contained fewer chiral centers and could be synthesized more cheaply. Nonetheless, one problem that must be overcome in chemical synthesis of a baminomycin is the extreme toxicity of the compound; great care and attendant expense must be taken to ensure that the active toxin is contained during synthesis. There would be significant advantages from a synthesis that did not require production of the highly toxic final compound.

Prodrugs have been investigated as a means to lower the unwanted toxicity or some other negative attribute of a drug without loss of efficacy. A prodrug is a drug that has been chemically modified to render it inactive (or less active) but that, subsequent to administration, is metabolized or otherwise converted to the active form of the drug in the body. For example, in an effort to improve drug targeting, prodrugs have been developed that are activated under hypoxic conditions. “Hypoxia” is a condition of low oxygen levels; most solid tumors larger than about 1 mm in diameter contain hypoxic regions (see the references Coleman, 1988, J. Nat. Canc. Inst. 80: 310; and Vaupel et al., Cancer Res. 49: 6449, each of which is incorporated herein by reference). Hypoxia creates a bioreductive environment, and certain anti-cancer agents have been converted into prodrugs that can be activated in such environments. See the reviews by de Groot et al., 2001, Current Medicinal Chem. 8: 1093-1122; Naylor et al., May 2001, Mini. Rev. Med. 1(1): 17-29; and Denny, 2001, Eur. J. Med. Chem. 36: 577-595, each of which is incorporated herein by reference.

As a tumor grows, it requires a blood supply and thus the growth of new vasculature. The new vasculature that supports tumor growth is often highly unordered, leaving significant portions of the tumor under-vascularized and subject to intermittent vascular blockage. The vascular architecture of the tumor can contribute significantly to the cancer's ability to survive drug therapy in at least two different ways. First, if the drug must reach the cancer through the bloodstream, then not as much drug will reach the under-vascularized, hypoxic areas of the tumor. Second, to the extent the drug requires oxygen to be effective, then the drug will be less effective in the hypoxic regions of the tumor. See the reference Stubbs et al., 2003, Current Molecular Medicine 3: 49-59, incorporated herein by reference.

Conversely, however, the hypoxic environment is conducive to reductive events that can be used to generate reduced derivatives of a variety of chemical groups (see the reference Workman et al., 1993, Cancer and Metast. Rev. 12: 73-82), and bioreductive prodrug compounds have been developed to exploit such environments. These prodrugs include the antibiotics Mitomycin C (MMC) and Porfiromycin (POR), N-oxides such as Tirapazamine (TRZ; see the reference Zeeman et al., 1986, Inst. J. Radiot. Oncol. Biol. Phys. 12: 1239), quinones such as the indoloquinone E09 (see the reference Bailey et al., 1992, Int. J. Radiot. Oncol. Biol. Phys. 22: 649), cyclopropamitosenes (EP-A-0868137), and a tertiary amine-N-oxide analogue of Mitoxantrone (AQ4N) that is activated by cytochrome P450 3A4 (see the references Patterson, 1993, Cancer Metast. Rev. 12: 119; and Patterson, 1994, Biochem. Pharm. Oncol. Res. 6: 533). Other bioreductively activated prodrug compounds include the nitroimidazole derivatives that have been reported to be useful in cancer radiotherapy as radio-sensitizing agents (see the patent publications EP312858 and WO91/11440) and potentiatiors of chemotherapeutic agents (see U.S. Pat. No. 4,921,963). Nitroimidazole has also been conjugated to the anti-cancer agent PARP 5-bromoisoquinolinone (see the reference Parveen et al., July 1999, Bioorg. Med. Chem. Lett., 9:2031-36). In addition, PCT publication WO 00/64864, incorporated herein by reference describes a wide variety of prodrug compounds that can be activated by a bacterial nitroreductase or by hypoxia. See also U.S. Pat. Nos. 5,780,585 and 5,977,065, each of which is incorporated herein by reference. The nitroimidazole moiety itself is, however, somewhat cytotoxic to normal cells, because it undergoes redox cycling and generates superoxides under oxygenated conditions.

Thus, there remains a need to provide drugs to treat cancer. Such drugs would be especially beneficial if they targeted cancer cells more effectively than current drugs and had fewer, less serious side effects, and were cost-effective to synthesize. The present invention helps meet this need.

### SUMMARY OF THE INVENTION

In one aspect, the present invention provides the following anthracycline compound of formula (I):

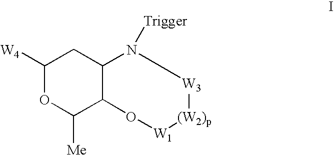

wherein
p is 1 or 2;

### W₁ is C(V₁)₂, C═O, or SO₂;

W₂ is C(V₁)₂, NV₁, O, or S with the proviso that when p is 2 both W₂ are not O;
W₃ is CV₁V₂ wherein each V₁ is independently H, C₁-C₆ alkyl or heteroalkyl and V₂ is hydrogen, hydroxy, mercapto, C₁-C₆ alkylthio and C₁-C₆ alkoxy;
W₄ is selected from the group consisting of:

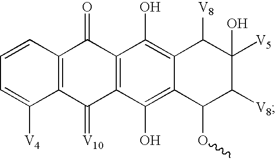

wherein V₄ is hydrogen, C₁-C₆ alkyl or heteroalkyl, hydroxy, C₁-C₆ alkoxy, amino, C₁-C₆ alkylamino, C₁-C₆ dialkylamino, mercapto, or C₁-C₆ alkylthio; V₅ is selected from the group consisting of —CH₂CH₃, —COCH₃, —CH(OH)CH₃, —COCH₂OH, —CH(OH)CH₂OH, —C(—N-Z₁)-CH₃, and —C(═N-Z₁)-CH₂OH wherein Z₁ is —OZ₂ or —N(Z₂)₂ wherein each Z₂ is selected from the group consisting of hydrogen, C₁-C₆-acyl or heteroacyl, aroyl or heteroaroyl, C₁-C₆ alkyl or heteroalkyl, and aryl or heteroaryl;
V₁₀ is O or NH;
each V₈ is halo or hydrogen provided that they are both not halo
   Trigger is —[C(Z₄)₂-Z₇]_{w}—(C(═O)—O)_{q}—[C(Z₄)₂-Z₅-Z₆]ᵤ—C(Z₄)₂[—C(Z₄)═C(Z₄)]₁-Z₃ or —[C(Z₄)₂-Z₇]_{w}—(S(═O)₂)_{q}—[C(Z₄)₂-Z₅-Z₆]ᵤ—C(Z₄)₂-[C(Z₄)═C(Z₄)]₁-Z₃,
wherein each w, q, u, and independently is 0 or 1; each Z₄ independently is hydrogen, halo, C₁-C₆ alkyl or heteroalkyl, aryl or heteroaryl, C₁-C₆ acyl or heteroacyl, aroyl, or heteroaroyl;
Z₃ is selected from the group consisting of:

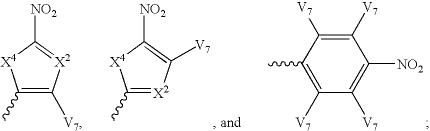

wherein X⁴ is NV¹, O, or S wherein V¹ is defined as before; each X² is N or CV⁷ wherein each V⁷ is alkyl, aryl, hydrogen, halogen, nitro, C₁-C₆ alkoxy, cyano, CO₂H, or CON(V₁)₂.
Z₅ is

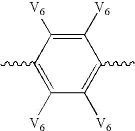

wherein each V₆ is hydrogen, halo, nitro, C₁-C₆ alkoxy, cyano, CO₂H, CON(V₁)₂;
Z₆ is S, O, or NV₁—(C(═O)—O)ᵥ wherein V₁ is defined as above and v is 0 or 1 provided that if v is 0, then Z₆ is other than NH;
Z₇ is S, O, or NV₁ provided that if Z₇ is S or O then q=0; and
   an individual isomer or a racemic or non-racemic mixture of isomers, a pharmaceutically acceptable salt, solvate, hydrate, or a prodrug thereof.

In another aspect, the present invention provides the anthracycline compound of formula (II):

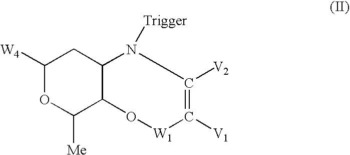

wherein W₁, W₄, V₁, V₂ and Trigger are defined as above; and
an individual isomer or a racemic or non-racemic mixture of isomers, a pharmaceutically acceptable salt, solvate, hydrate, or a prodrug thereof.

In another aspect, the present invention provides compounds that comprise a selective prodrug Trigger and cyclic anthracycline toxin or supertoxin. The Trigger can be a nitroimidazole, a hypoxia or bacterial nitroreductase activateable Trigger, or can be a moiety acted upon by a tumor specific condition, such as a peptide cleaved by a tumor specific protease or a sugar cleaved by a glycosidase. In some compounds of the invention, the cyclic anthracycline toxin or supertoxin can be conjugated via the linker to a tumor specific antibody, which is endocytosed. Conventional disulfide release can be the triggering event. In one embodiment, the prodrug has a “fuse” or spacer between the hypoxic activator (Z₃) and the toxin or supertoxin, which allows for the activated prodrug to diffuse from the site of trigger release before complete activation of the toxin or supertoxin. In one embodiment, this fuse has a phenolic ether linkage.

In one embodiment, the compound released upon reduction of the hypoxic activator may have an IC₅₀ of less than about 100 nM.

In one aspect, the protected cyclic anthracycline toxin may be used for treating cancer by administering to a subject a therapeutically effective amount of a protected protected cyclic anthracycline toxin. In these methods, the protected cyclic anthracycline toxin may be administered alone or in combination with an effective amount of one or more chemotherapeutic agents, an effective amount of radiotherapy, a surgery procedure, or any combination of the foregoing. Chemotherapeutic agents that may be used are described in detail in the Detailed Description section.

In one embodiment, cancers that may be treated are described in detail in the Detailed Description section and include lung cancer, non-small cell lung cancer, breast cancer, colon cancer, head and neck cancer, ovarian cancer, pancreatic cancer, and prostate cancer.

### BRIEF DESCRIPTION OF THE DRAWINGS

The foregoing and other objects, features and advantages of the present invention will be more readily understood upon reading the following detailed description in conjunction with the drawings as described hereinafter.

FIG. 1 is a graph illustrating the dose response profile for compounds of the invention (2a, 2b and 2c) as compared to Daunorubicin under normoxic conditions (normoxia) and hypoxic conditions (hypoxia) as determined by fraction of surviving cells.

FIG. 2 is a graph illustrating the dose response profile for Daunorubicin under normoxic conditions (normoxia) and hypoxic conditions (hypoxia) as determined by fraction of surviving cells.

FIG. 3 is a graph illustrating the dose response profile for compounds of the invention (2d and 2e) under normoxic conditions (normoxia) and hypoxic conditions (hypoxia) as determined by fraction of surviving cells.

### DETAILED DESCRIPTION OF THE INVENTION

In this detailed description are first provided definitions useful in understanding the compounds, compositions, and methods described in this patent. A general description of prodrug compounds (protected cyclic anthracycline toxins) that may be used for treating cancer is then provided, followed by descriptions of various components in these compounds and methods of making the compounds. A description of methods of treatment using the compounds, including a description of cancers that may be treated, is then included, followed by descriptions of formulations, modes of delivery, dosages, etc that may be used with the compounds and methods described in the patent. Combination therapies, in which the compounds described herein are used in combination with other treatments, and finally examples are provided of the compounds, compositions, and methods described herein.

### DEFINITIONS

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, “a” or “an” means “at least one” or “one or more.”

“Alkyl” refers to a linear saturated monovalent hydrocarbon radical or a branched saturated monovalent hydrocarbon radical having the number of carbon atoms indicated in the prefix. For example, (C₁-C₈)alkyl is meant to include methyl, ethyl, n-propyl, 2-propyl, n-butyl, 2-butyl, tert-butyl, pentyl, and the like. For each of the definitions herein (e.g., alkyl, alkenyl, alkoxy, araalkyloxy), when a prefix is not included to indicate the number of main chain carbon atoms in an alkyl portion, the radical or portion thereof will have six or fewer main chain carbon atoms. (C₁-C₆) alkyl may be further substituted with substituents, including for example, hydroxy, amino, mono or di(C₁-C₆)alkyl amino, halo, C₂-C₆ alkenyl ether, cyano, nitro, ethenyl, ethynyl, C₁-C₆ alkoxy, C₁-C₆ alkylthio, —COOH, —CONH₂, mono- or di-(C₁-C₆)alkyl-carboxamido, —SO₂NH₂, —OSO₂—(C₁-C₆)alkyl, mono or di(C₁-C₆) alkylsulfonamido, aryl and heteroaryl.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆)alkenyl is meant to include, ethenyl, propenyl, 1,3-butadienyl and the like.

“Aryl” refers to a monovalent monocyclic or bicyclic aromatic hydrocarbon radical of 6 to 10 ring atoms which is substituted independently with one to four substituents, preferably one, two, or three substituents selected from alkyl, cycloalkyl, cycloalkylalkyl, halo, nitro, cyano, hydroxy, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{y} together is cycloalkyl or heterocyclyl. More specifically the term aryl includes, but is not limited to, phenyl, biphenyl, 1-naphthyl, and 2-naphthyl, and the substituted forms thereof.

“Cycloalkyl” refers to a monovalent cyclic hydrocarbon radical of three to seven ring carbons. The cycloalkyl group may have one double bond and may also be optionally substituted independently with one, two, or three substituents selected from alkyl, optionally substituted phenyl, or —C(O)R^{z} (where R^{z} is hydrogen, alkyl, haloalkyl, amino, mono-alkylamino, di-alkylamino, hydroxy, alkoxy, or optionally substituted phenyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonyl-cyclohexyl, and the like.

“Heteroalkyl” means an alkyl radical as defined herein with one, two or three substituents independently selected from cyano, —OR^{w}, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, aralkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that n is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, halo, fluoro, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{X}R^{y}, or —S(O)ₚR^{z} portions.

“Heteroaryl” means a monovalent monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to four substituents, preferably one or two substituents, selected from alkyl, cycloalkyl, cycloalkyl-alkyl, halo, nitro, cyano, hydroxy, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)n—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is cycloalkyl or heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl and the derivatives thereof. Unless indicated otherwise, the arrangement of the hetero atoms within the ring may be any arrangement allowed by the bonding characteristics of the constituent ring atoms.

“Heterocyclyl” or “cycloheteroalkyl” means a saturated or unsaturated non-aromatic cyclic radical of 3 to 8 ring atoms in which one to four ring atoms are heteroatoms selected from O, NR (where R is independently hydrogen or alkyl) or S(O)ₚ (where p is an integer from 0 to 2), the remaining ring atoms being C, where one or two C atoms may optionally be replaced by a carbonyl group. The heterocyclyl ring may be optionally substituted independently with one, two, or three substituents selected from alkyl, aryl, arylalkyl, heteroaryl, heteroarylalkyl, cycloalkyl, cycloalkylalkyl, halo, nitro, cyano, hydroxy, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, pyridyl, tetrahydropyranyl, N-methylpiperidin-3-yl, N-methylpyrrolidin-3-yl, 2-pyrrolidon-1-yl, furyl, quinolyl, thienyl, benzothienyl, pyrrolidinyl, piperidinyl, morpholinyl, pyrrolidinyl, tetrahydrofuranyl, tetrahydrothiofliranyl, 1,1-dioxo-hexahydro-1λ⁶-thiopyran-4-yl, tetrahydroimidazo[4,5-c]pyridinyl, imidazolinyl, piperazinyl, and piperidin-2-onyl and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms.

The terms “optional” or “optionally” as used throughout the specification mean that the subsequently described event or circumstance may, but need not, occur, and that the description includes instances where the event or circumstance occurs and instances in which it does not. For example, “heterocyclo group optionally mono- or di-substituted with an alkyl group means that the alkyl may, but need not be, present, and the description includes situations where the heterocyclo group is mono- or disubstituted with an alkyl group and situations where the heterocyclo group is not substituted with an alkyl group.

“Optionally substituted” means a ring which is optionally substituted independently with substituents.

A combination of substituents or variables is permissible only if such a combination results in a stable or chemically feasible compound. A stable compound or chemically feasible compound is one in which the chemical structure is not substantially altered when kept at a temperature of 4° C. or less, in the absence of moisture or other chemically reactive conditions, for at least a week.

As used herein, a “prodrug” is a compound that, after administration, is metabolized or otherwise converted to an active or more active form with respect to at least one biological property, relative to the pharmaceutically active compound. To produce a prodrug, a pharmaceutically active compound (or a suitable precursor thereof) is modified chemically such that the modified form is less active or inactive, but the chemical modification is effectively reversible under certain biological conditions such that a pharmaceutically active form of the compound is generated by metabolic or other biological processes. A prodrug may have, relative to the drug, altered metabolic stability or transport characteristics, fewer side effects or lower toxicity, or improved flavor, for example (see the reference Nogrady, 1985, Medicinal Chemistry A Biochemical Approach, Oxford University Press, New York, pages 388-392). Prodrugs can also be prepared using compounds that are not drugs but which upon activation under certain biological conditions generate a pharmaceutically active compound. As used herein a protected cyclic anthracyclin toxin is a prodrug that upon activation releases a modified cyclic anthracyclin toxin or the active anthracyclin toxin.

As used herein, an “anti-neoplastic agent”, “anti-tumor agent”, or “anti-cancer agent”, refers to any agent used in the treatment of cancer. Such agents can be used alone or in combination with other compounds and can alleviate, reduce, ameliorate, prevent, or place or maintain in a state of remission of clinical symptoms or diagnostic markers associated with neoplasm, tumor or cancer. Anti-neoplastic agents include, but are not limited to, anti-angiogenic agents, alkylating agents, antimetabolite, certain natural products, platinum coordination complexes, anthracenediones, substituted ureas, methylhydrazine derivatives, adrenocortical suppressants, certain hormones and antagonists, anti-cancer polysaccharides and certain herb or other plant extracts.

As used herein, an “protected cyclic anthracycline toxin treatment,” “anti-neoplastic treatment” “cancer therapy,” “cancer treatment,” or “treatment of cancer,” refers to any approach for ameliorating the symptoms of or delaying the progression of a neoplasm, tumor, or cancer by reducing the number of or growth of cancer cells in the body, typically (but not limited to) by killing or halting the growth and division of cancer cells.

As used herein a “cytotoxic agent” is an agent that produces a toxic effect on cells. As used herein a “cytostatic agent” is an agent that inhibits or suppresses cellular growth and multiplication.

As used herein, a “bioreductive compound” refers to a compound that accepts electrons in an oxidation-reduction reaction.

As used herein, “cancer” refers to one of a group of more than 100 diseases caused by the uncontrolled growth and spread of abnormal cells that can take the form of solid tumors, lymphomas, and non-solid cancers such as leukemia.

As used herein, “malignant” refers to cells that have the capacity of metastasis, with loss of both growth and positional control.

As used herein, “neoplasm” (neoplasia) or “tumor” refers to abnormal new cell or tissue growth, which may be benign or malignant.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of cancer, diminishment of extent of disease, delay or slowing of disease progression, amelioration, palliation or stabilization of the disease state, and other beneficial results described below.

As used herein, “reduction” of a symptom or symptoms (and grammatical equivalents of this phrase) means decreasing of the severity or frequency of the symptom(s), or elimination of the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “therapeutically effective amount” of a drug is an amount of a drug that, when administered to a subject with cancer, will have the intended therapeutic effect, e.g., alleviation, amelioration, palliation or elimination of one or more manifestations of cancer in the subject. The full therapeutic effect does not necessarily occur by administration of one dose, and may occur only after administration of a series of doses. Thus, a therapeutically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

In one aspect, the present invention provides the following Anthracycline compound of formula (I):

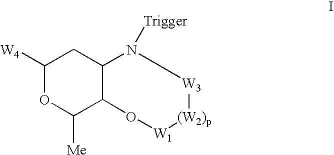

wherein
p is 1 or 2;

### W₁ is C(V₁)₂, C═O, or SO₂;

W₂ is C(V₁)₂, NV₁, O, or S with the proviso that when p is 2 both W₂ are not 0;
W₃ is CV₁V₂ wherein each V₁ is independently H, C₁-C₆ alkyl or heteroalkyl and V₂ is hydrogen, hydroxy, mercapto, C₁-C₆ alkylthio or C₁-C₆ alkoxy;

### W₄ is:

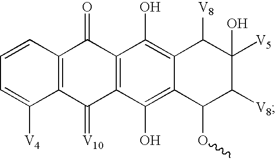 [0063] wherein V₄ is hydrogen, C₁-C₆ alkyl or heteroalkyl, hydroxy, C₁-C₆ alkoxy, amino, C₁-C₆ alkylamino, C₁-C₆ dialkylamino, mercapto, or C₁-C₆ alkylthio; V₅ is selected from the group consisting of —CH₂CH₃, —COCH₃, —CH(OH)CH₃, —COCH₂OH, —CH(OH)CH₂OH, —C(═N-Z₁)-CH₃, and —C(═N-Z₁)-CH₂OH wherein Z₁ is —OZ₂ or —N(Z₂)₂ wherein each Z₂ is selected from the group consisting of hydrogen, C₁-C₆-acyl or heteroacyl, aroyl or heteroaroyl, C₁-C₆ alkyl or heteroalkyl, and aryl or heteroaryl;
V₁₀ is O or NH;
each V₈ is halo or hydrogen provided that they are both not halo
   Trigger is —[C(Z₄)₂-Z₇]_{w}—(C(═O)—O)_{q}—[C(Z₄)₂-Z₅-Z₆]ᵤ—C(Z₄)₂[—C(Z₄)═C(Z₄)]₁-Z₃ or —[C(Z₄)₂-Z₇]_{w}—(S(═O)₂)_{q}—[C(Z₄)₂-Z₅-Z₆]ᵤ—C(Z₄)₂-[C(Z₄)═C(Z₄)]₁-Z₃,
wherein each w, q, u, and independently is 0 or 1; each Z₄ independently is hydrogen, halo, C₁-C₆ alkyl or heteroalkyl, aryl or heteroaryl, C₁-C₆ acyl or heteroacyl, aroyl, and heteroaroyl;
Z₃ is selected from the group consisting of:

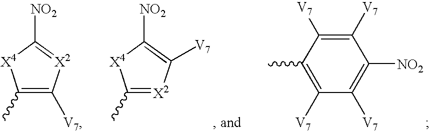

wherein X⁴ is NV¹, O, or S wherein V¹ is defined as before; each X² is N or CV⁷ wherein each V⁷ is alkyl, aryl, hydrogen, halogen, nitro, C₁-C₆ alkoxy, cyano, CO₂H, or CON(V₁)₂.
Z₅ is

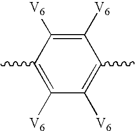

wherein each V₆ is hydrogen, halo, nitro, C₁-C₆ alkoxy, cyano, CO₂H, or CON(V₁)₂;
Z₆ is S, O, or NV₁—(C(═O)—O)ᵥ wherein V₁ is defined as above and v is 0 or 1 provided that if v is 0, then Z₆ is other than NH;
Z₇ is S, O, or NV₁ provided that if Z₇ is S or O then q=0; and
   an individual isomer or a racemic or non-racemic mixture of isomers, a pharmaceutically acceptable salt, solvate, hydrate, or a prodrug thereof.

In another aspect, the present invention provides the anthracycline compound of formula (II):

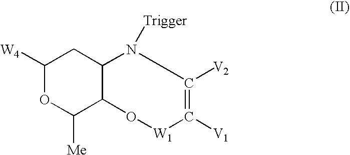

wherein W₁, W₄, V₁, V₂ and Trigger are defined as above; and
an individual isomer or a racemic or non-racemic mixture of isomers, a pharmaceutically acceptable salt, solvate, hydrate, or a prodrug thereof.

In one embodiment Z₃ is a substituted nitrobenzyl moiety. In one embodiment Z₃ is a substituted 4-nitrobenzyl moiety. In one embodiment Z₃ is a 4-nitrobenzyl moiety substituted with substituents selected from nitro, CO₂H, acyl, halo, and CON(V₁)₂ wherein V₁ is defined as before.

In one embodiment, in a compound of Formula (I) W₄ is:

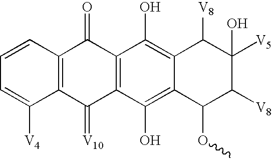

wherein
V₄ is hydrogen, methoxy, or hydroxy;
V₅ is —CH₂CH₃, —COCH₃, —CH(OH)CH₃, —COCH₂OH, —CH(OH)CH₂OH, —C(═N—NHCOPh)—CH₃, or —C(═N—NHCOPh)—CH₂OH;

### V₁₀ is O or NH; and

V₈ is hydrogen or fluoro.

In one embodiment, the present invention provides compounds of formulas (I) and (II) wherein Z₃ is selected from the group consisting of:

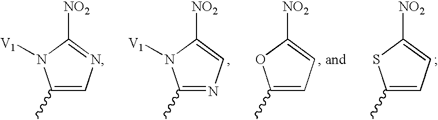

wherein V₁ is defined as before.

In one embodiment, the hypoxic activator (Z3) is capable of being reduced under hypoxic conditions but not under normoxic conditions. In one embodiment, when Z3 is reduced under hypoxic conditions, the Trigger is activated to release the cyclic anthracycline toxin or the modified cyclic anthracycline toxin Examples of hypoxia activators include, but are not limited to, for example, groups based on electron deficient nitrobenzenes, electron deficient nitrobenzoic acid amides, nitroazoles, nitroimidazoles, nitrothiophenes, nitrothiazoles, nitrooxazoles, nitrofurans, and nitropyrroles, where each of these classes of moieties may be substituted or unsubstituted, such that the redox potential for the group lies within a range where the group can undergo reduction in the hypoxic conditions of a tumor. One of skill in the art will understand, in view of the description herein, how to substitute these and other hypoxia labile protecting groups to provide a redox potential that lies within said range.

Generally, one of skill in the art can “tune” the redox potential of a hypoxia activator (Z3) by modifying that group to contain electron withdrawing groups, electron donating groups, or some combination of such groups. For example, nitrothiophene, nitrofuranfuran, and nitrothiazole groups may be substituted with one or more electron donating groups, including but not limited to methyl, methoxy, or amine groups, to achieve the desired redox potential. In another example, the nitropyrrole moiety can be substituted with an electron withdrawing group, including but not limited to cyano, carboxamide, —CF₃, and sulfonamide groups, to achieve the desired redox potential. For this purpose, strong electron withdrawing groups such as cyano, sulfone, sulfonamide, carboxamide, or —CF₃, and milder electron withdrawing groups such as —CH₂-halogen, where halogen is —F, —Cl, or —Br, can be used.

In one embodiment -Z₅-Z₆- together is selected from the group consisting of:

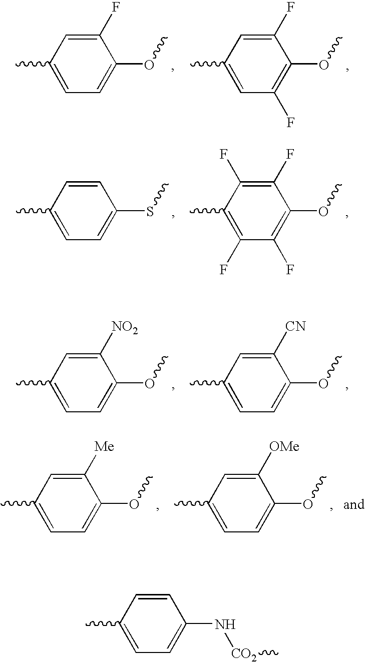

In one embodiment, the present invention provides a compound of formula:

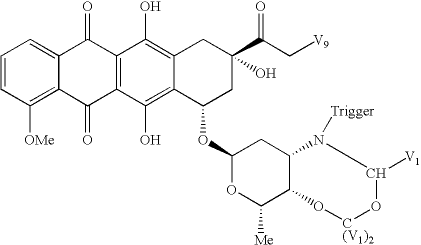

wherein each V₁ is C₁-C₆ alkyl or heteroalkyl and V₉ is H or OH.

In a related embodiment, the present invention provides a compound of formula:

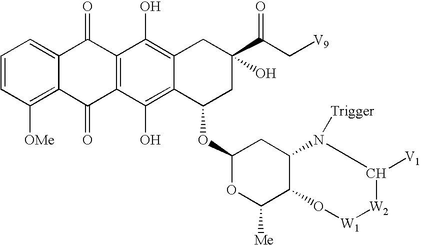

wherein W₁ is CO or SO₂; W₂ is NV₁ wherein each V₁ is C₁-C₆ alkyl or heteroalkyl; and V₉ is H or OH.

In one embodiment, the present invention provides a compound of formula:

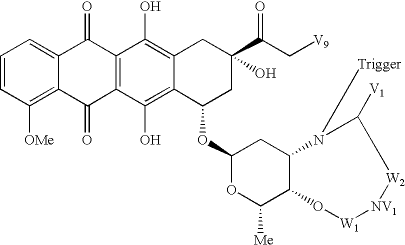

wherein W₁ is CO or SO₂; W₂ is NV₁ or O wherein each V₁ is C₁-C₆ alkyl or heteroalkyl; and V₉ is H or OH.

In a related embodiment, the present invention provides a compound of formula:

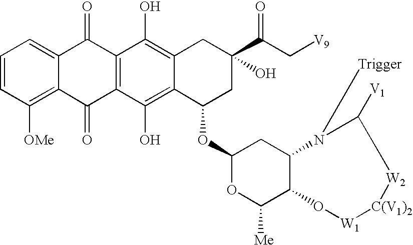

wherein W₁ is CO or SO₂; W₂ is NV₁, O, or S wherein each V₁ is hydrogen, C₁-C₆ alkyl or heteroalkyl, C₁-C₆ alkoxy, C₁-C₆ thioalkyl, hydroxy, or mercapto; and V₉ is H or OH.

In a related embodiment, the present invention provides a compound of formula:

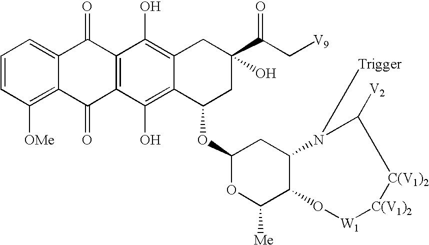

wherein W₁ is CO or SO₂; V₁ and V₂ are defined as in formula (I); and V₉ is H or OH.

In a related embodiment, the present invention provides a compound of formula:

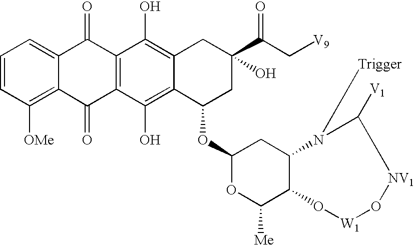

wherein W₁ is CO or SO₂; V₁ is defined as in formula (I); and V₉ is H or OH.

In another embodiment, the present invention provides a compound of formula:

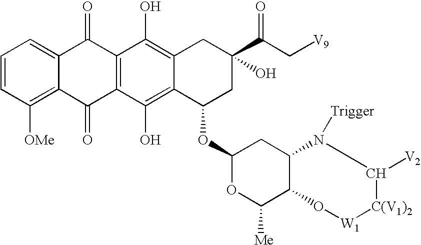

wherein W₁ is CO or SO₂; V₁ and V₂ are defined as in formula (I); and V₉ is H or OH.

In another embodiment, the present invention provides a compound of formula

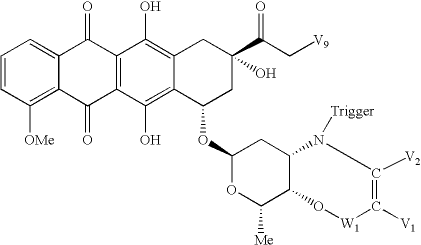

wherein W₁ is CO or SO₂; V₁ and V₂ are defined as in formula (II); and V₉ is H or OH.

In one embodiment, C₁-C₆ alkyl groups above can be appended with groups which improve solubility, biodistribution, or cellular permeation.

In one embodiment the present invention provides the compounds of the formula:

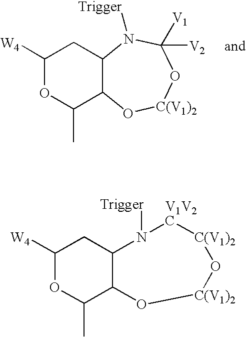

wherein W₄, V₁, V₂, and Trigger are defined as above.

In one embodiment, the present invention provides the compounds:

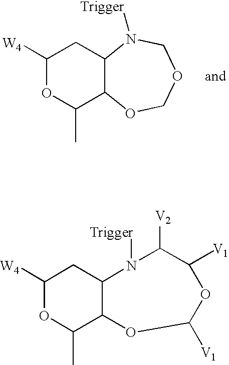

wherein W₄, V₁, V₂, and Trigger are defined as above

In one embodiment, the present invention provides the following compounds:

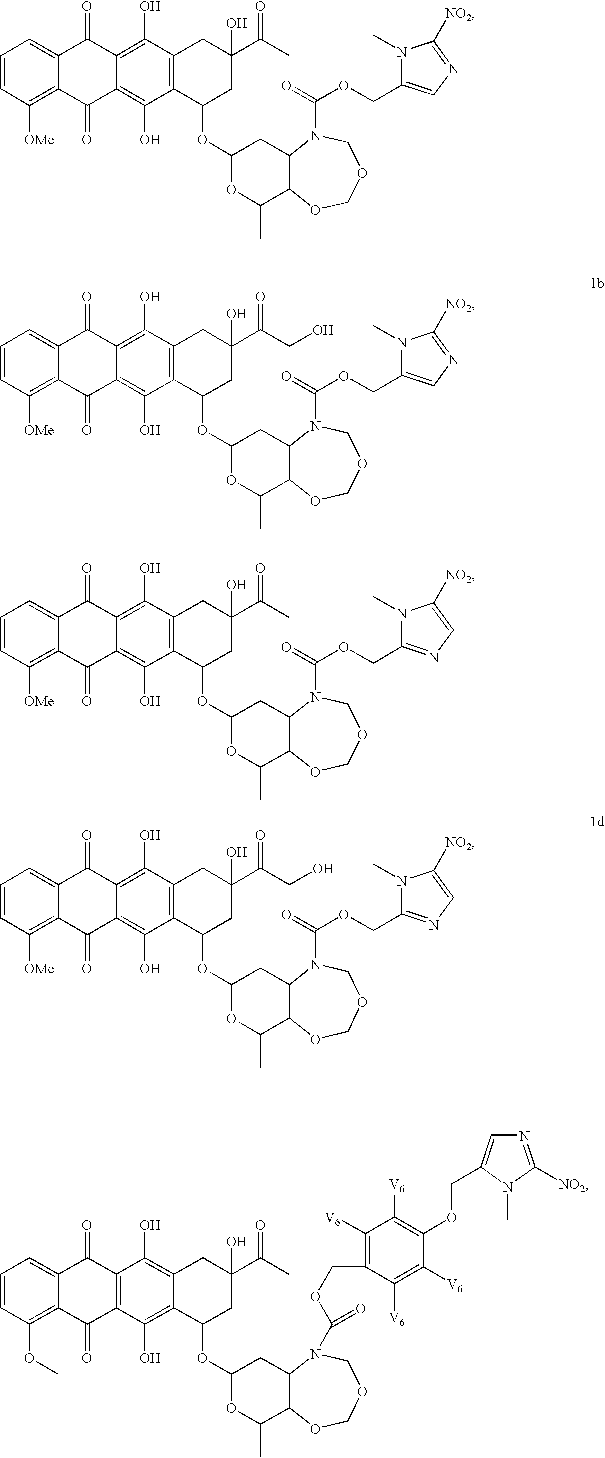

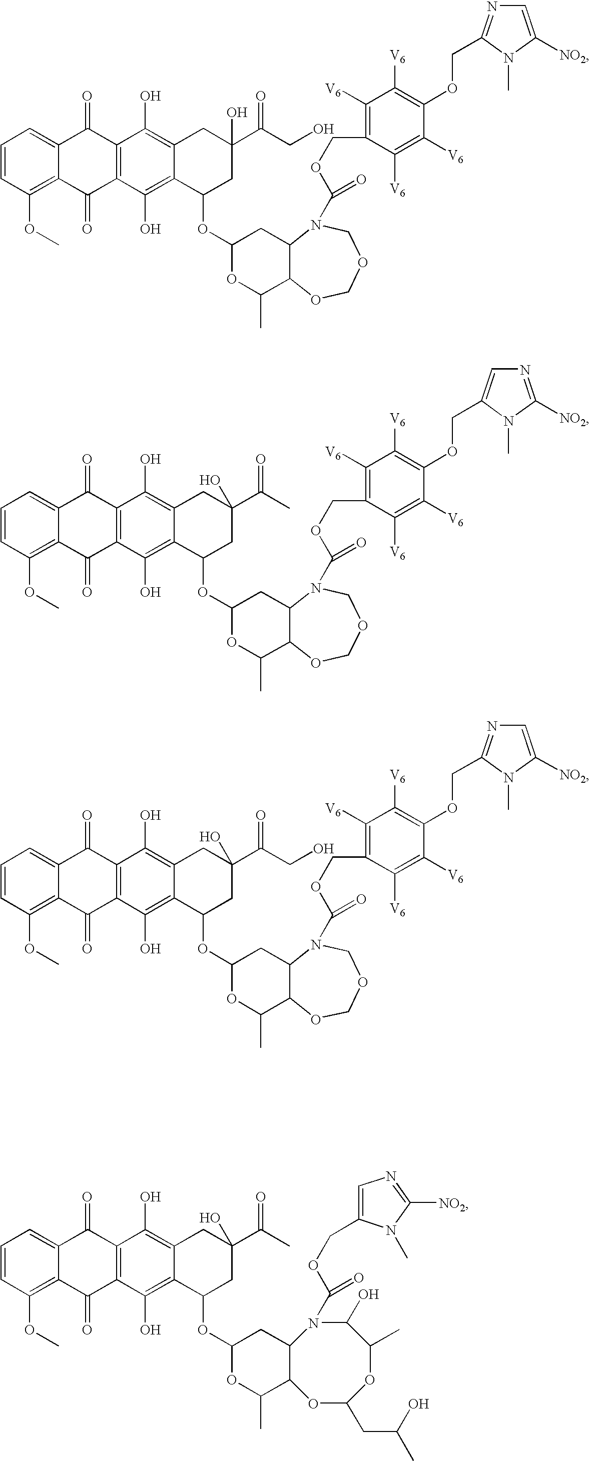

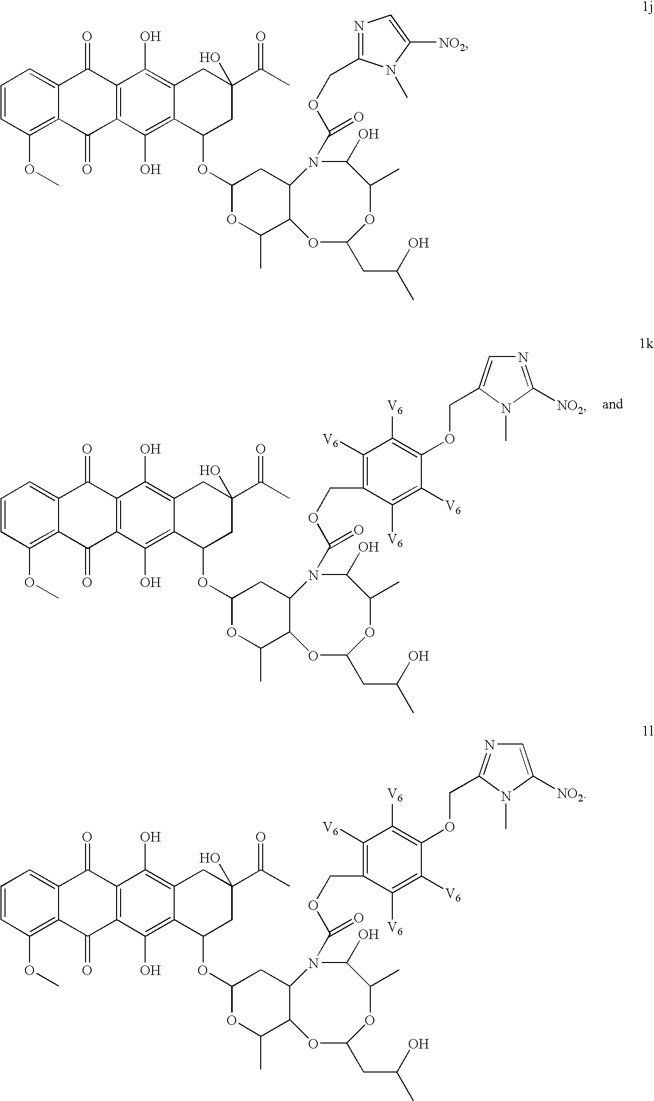

wherein each V₆ independently is fluoro or hydrogen.

In another embodiment, the present invention provides compounds useful in the treatment of cancer, wherein such compounds are selected from the group of compounds defined by the Formulas I and II.

In one embodiment, the compounds of the invention are prodrugs of baminomycin analogs. As noted in the background section above, super toxic daunorubicin analogs referred to as bamrinomycin analogs include natural products derived from fermentation. The baminomycins contains an 8 membered ring with a hydroxyaminal moiety as a precursor to an alkylating imine group. Crosslinking properties and background information on baminomycin are contained in the reference Perrin et al., supra.

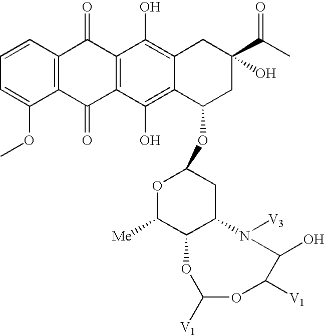

wherein V₁ is defined as in Formula I and V₃ is C₁-C₆ alkyl or heteroalkyl, acyl, aminoacyl, aroyl, or hetroaroyl.

### Methods of Making the Protected Cyclic Anthracyclinte Toxins

The protected cyclic anthracycline toxins described herein may be made by a variety of methods. Given the synthesis methods described in the examples below and their knowledge of synthetic medicinal chemistry, one of skill in the art will be able to synthesize the protected cyclic anthracycline toxins in a straightforward manner.

In one embodiment the present invention provides methods of synthesis of the compounds of the invention. Anthracyclines useful in the synthesis of compounds (1) and (II) are described in the references Monneret et al., Eur. J. Med. Chem., 2001, 36, 483-93 and Anthracycline Antibiotics: New Analogues, Methods of Delivery, and Mechanisms of Action, 1995, Ed. Waldemar Priebe, Oxford University Press, and also further below.

In a related embodiment the synthesis uses as a starting material a compound of formula (III):

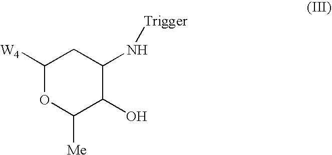

wherein W₄ and Trigger are defined as above. Synthesis of a compound of formula (III) can be performed by adapting methods known to one of skill in the art. For example, the commonly assigned reference Matteucci et al, PCT Publication No. WO 04/87075 describes synthetic methods which can be adapted for the synthesis of (III). The present invention provides methods for cyclization joining the NH-Trigger group and the OH group in (III) yields compound (I) as provided below in Schemes 1-VII.

Various baminomycins have been isolated, with the various analogs differing with respect to the R₁ and R₂ groups shown in the structure above. All natural baminomycins have R being a hydrogen. Synthetic baminomycin or analogs derived from commonly available daunorubicin have not been reported.

In one embodiment, the present invention provides easy to synthesize baminomycin analogs that are masked as biologically activateable prodrugs. In one embodiment, the present invention provides methods for making such biologically activateable prodrugs. One important aspect of this invention is that the 8 membered ring that confers super toxicity to the unmasked molecule can be introduced during synthesis in accordance with certain methods of the invention after the blocking prodrug group has been installed on the 3′ amino group of daunorubicin (or doxorubicin). This precludes having to work with a native highly toxic baminomycin analog where R═H. The baminomycin prodrugs of the invention include doxorubicin and duanorubicin analogs linked via a carbamate, sulfonamide, aminal or alkyl connection within a Trigger moiety. In one embodiment, the Trigger moiety contains a nitroimidazole Triggering moiety, as shown below in Scheme I. The compounds provided in Scheme I can be further reacted to yield compound of Formula (I) of the present invention.

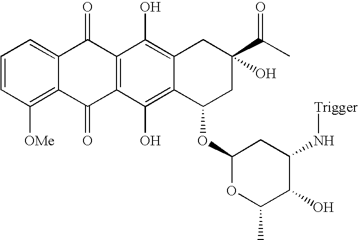

wherein Trigger is:

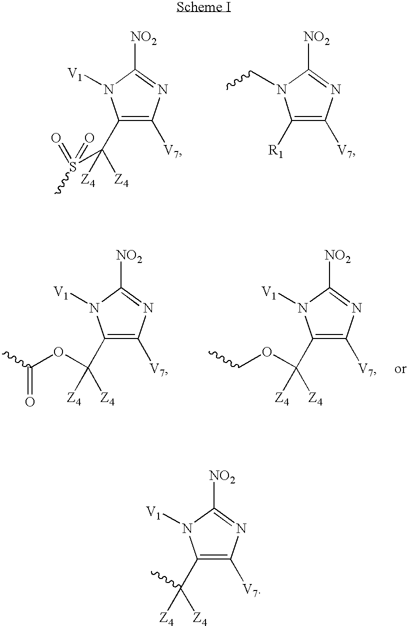

wherein Z₄, V₁, and V₇ are defined as in Formula I.

The Trigger moiety shown in Scheme I above is a nitroimidazole derivative that can be released intracellularly under low oxygen conditions and so targets the hypoxic zones of tumors. Other nitroimidazole substitution patterns and other nitroazole Triggers that are hypoxically activated can also serve as the Trigger in the prodrug compounds of the invention.

In another embodiment, the present invention provides baminomycin analogs and methods for their synthesis in which a latent aldehyde is masked as a 1,2 diol for eventual oxidation to the aldehyde. This embodiment of the invention is illustrated in the reaction Scheme (II) provided below.

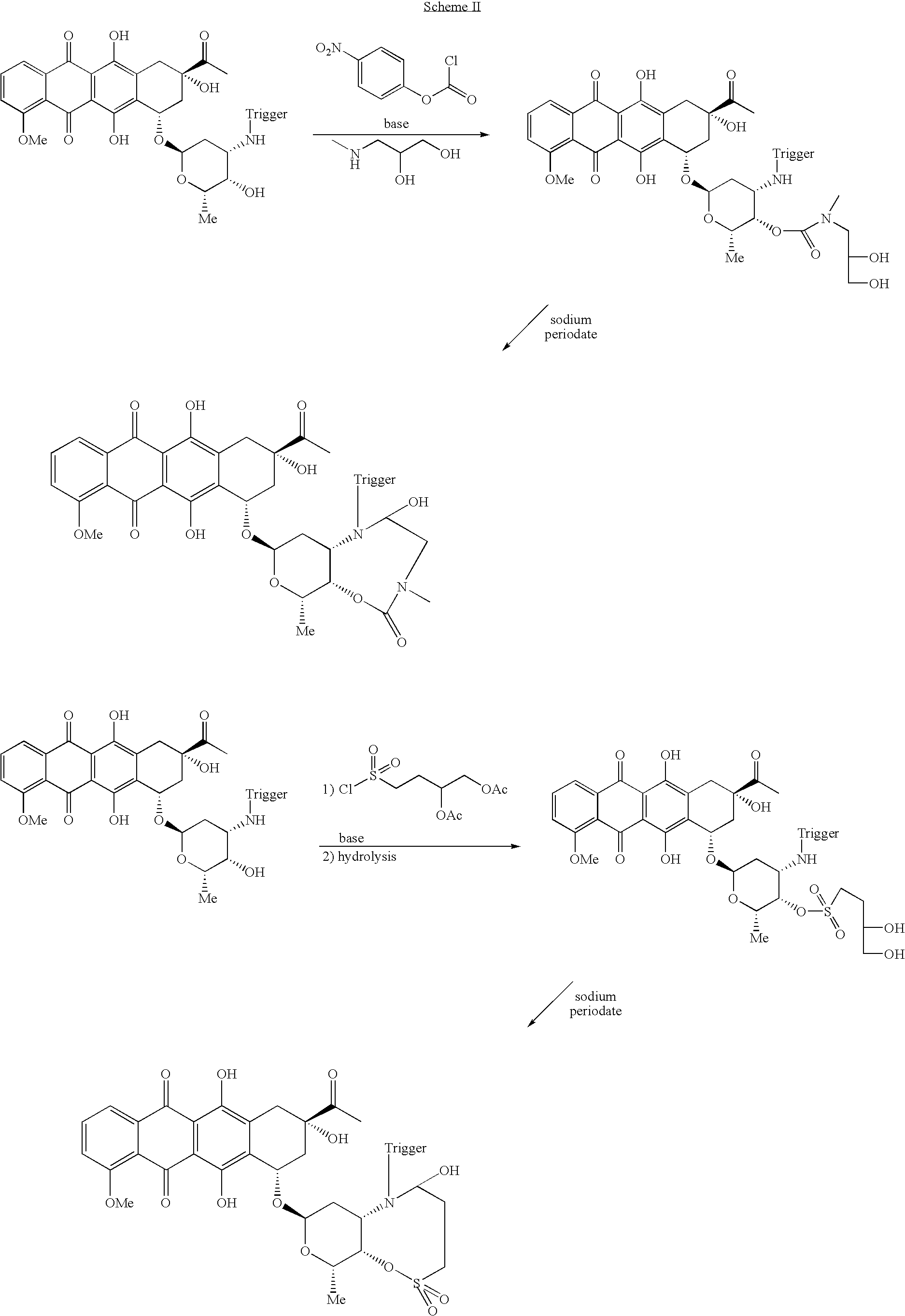

In another embodiment, the present invention provides methods for synthesizing compounds of the invention as provided below in Scheme III.

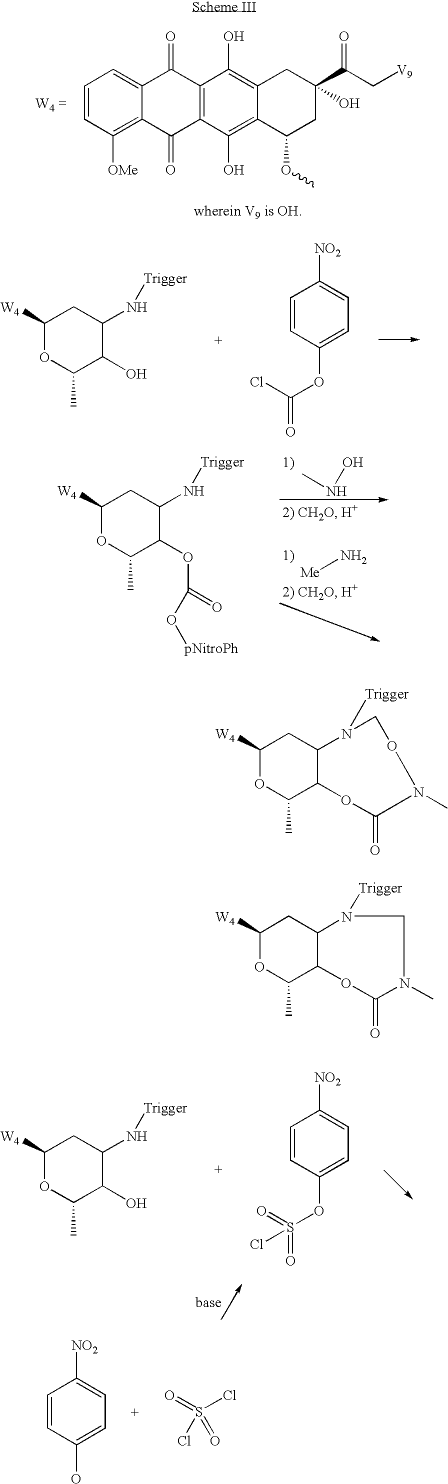

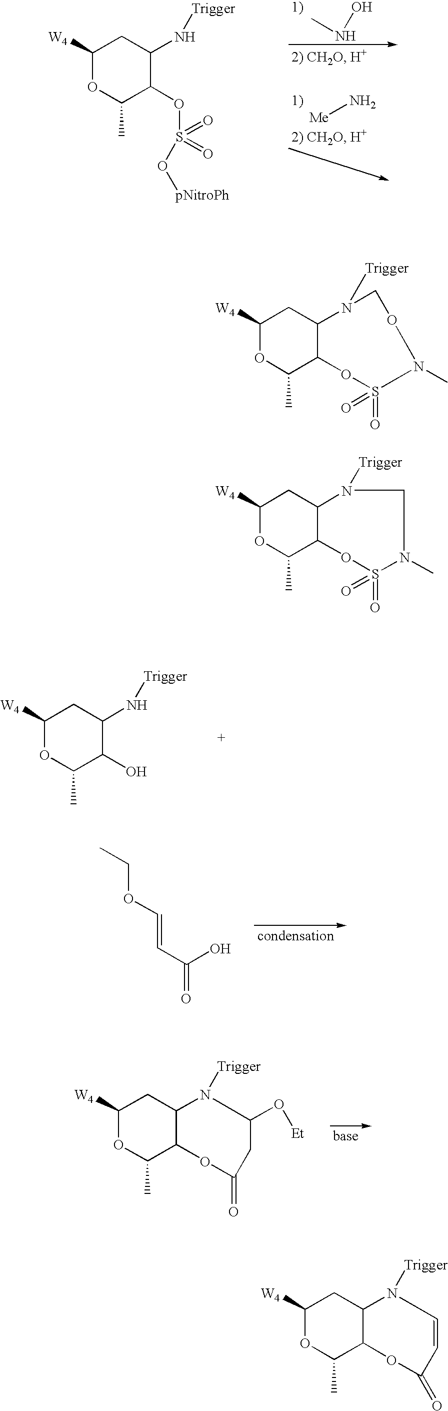

One of skill in the art will recognize that teaching relevant for making Triggers useful in the present invention are provided for example in the Matteucci et al. reference (supra, incorporated herein by reference). General methods of synthesizing hypoxia activated prodrugs and the use of hypoxia activated prodrugs in cancer treatment are provided in co-pending U.S. Application Nos. 60/629,723; 60/612,383; and 60/630,422 (all Matteucci et al., each of which is incorporated herein by reference). In one embodiment, the present invention provides a method of synthesizing compounds of Formulas I and II by modifying methods described in the references DeGroot et al., US Patent Publication No. 2004/0121,940; Davis et al., PCT Publication Nos. WO 04/85421 and WO 04/85361 (each of which is incorporated herein by reference).

Protected Cyclic Anthracycline Toxins can Release Cyclic Anthracycline Toxins or Modified Cyclic Anthracycline Toxins, Including “Super Toxins”: Depending on how the cyclic anthracycline toxin of Formulas I and II are bonded to the hypoxic activator (Z₃) and depending on the nature of the linking group, the fuse, or the spacer within the Trigger, the molecule released upon reduction of the hypoxic activator (Z₃) is either the cyclic anthracycline toxin or a modified cyclic anthracycline toxin that includes some or all of the linking group attached to the cyclic anthracycline toxin. In one embodiment, the present invention provides a linking group, a linker, a fuse, or a spacer having the Formula:—

—[C(Z₄)₂-Z₇]_{w}—(C(═O)—O)_{q}—[C(Z₄)₂-Z₅-Z₆]ᵤ—C(Z₄)₂[—C(Z₄)═C(Z₄)]₁— or

—[C(Z₄)₂-Z₇]_{w}—(S(═O)₂)_{q}—[C(Z₄)₂-Z₅-Z₆]ᵤ—C(Z₄)₂[—C(Z₄)═C(Z₄)]₁—

wherein w, q, u, w, Z₄, Z₅, and Z₆ is defined as in Formulas I and II.

In one embodiment, the present invention provides a compound which demonstrates a bystander effect upon activation under hypoxia because of the incorporation of a linking group, a fuse, or a spacer as described above. The bystander effect allows the modified cyclic anthracycline toxin of the present invention to diffuse or move into tumor zones which are not hypoxic enough to activate the prodrug compounds of the invention but reside nearby the hypoxic tumor zone which can activate these prodrugs.

That the molecule released upon activation of the Trigger may be different from the cyclic anthracycline toxin being protected will be appreciated by those of skill in the art. As used herein, a “modified cyclic anthracycline toxin” refers to a species that is released from a protected cyclic anthracycline toxin (i.e. a prodrug) and that is different from the cyclic anthracycline toxin itself. For example, a protected cyclic anthracycline toxin with Formula (I) or (II) may yield a modified cyclic anthracycline toxin upon reduction of the hypoxic activator (Z₃). When reduction of the hypoxic activator liberates a modified cyclic anthracycline toxin, the linking group attached to the cyclic anthracycline toxin may undergo rearrangement or degradation to yield either the unmodified cyclic anthracycline toxin or some other modified cyclic anthracycline toxin.

The protected cyclic anthracycline toxins described herein generally exhibit greater efficacy and/or fewer side effects than prior compounds. For example, certain protected cyclic anthracycline toxins described herein are conjugated to, or are activated by hypoxic conditions to release very powerful cytotoxic agents, “super toxins” with IC₅₀ values of less than 100 nM against a majority of the cancer cell lines in the NCI tumor cell line panel. Regarding possible toxicity of the protected cyclic anthracycline toxins, even though the protected cyclic anthracycline toxins still generate the superoxide that may cause unwanted side effects, those side effects are greatly reduced relative to the effects of prior compounds, because, on a molar basis, much less protected cyclic anthracycline toxin has to be given due to the highly cytotoxic nature of the anti-cancer agent released by the protected cyclic anthracycline toxin. Generally (maybe with the exception of compounds described in A 2-NITROIMIDAZOLE CARBAMATE PRODRUG OF 5-AMINO-1-(CHLOROMETHYL)-3-[5,6,7-TRIMETHOXYINDOL-2-YL)CARBONYL]-1,2-DIHYDRO-3H-BENZ[E]INDOLE (AMINO-SECO-CBI-TMI) FOR USE WITH ADEPT AND GDEPT, M. P. Hay et al., Bioorganic & Medicinal Chemistry Letters 9 (1999) 2237-2242, and PCT publication WO 00/64864) the protected cyclic anthracycline toxins described herein that release “super toxins” can be used in much lower doses than the nitroimidazole prodrugs heretofore known. These lower doses produce less superoxide (see discussion below) in normoxic tissue.

The protected cyclic anthracycline toxins can be used to release a wide variety of cyclic anthracycline toxins as is described infra.

Protected Cyclic Anthracycline Toxins may have reduced Toxicity: The protected cyclic anthracycline toxins, relative to the drugs to which they are converted in vivo, may be much less (at least ten and up to one million-fold less) toxic. The reduced toxicity results from a modification at the site of attachment of the Trigger (as in the case where activation of the protected cyclic anthracycline toxins releases the same cytotoxic agent that was used in the synthesis of the drug) or from the generation of a moiety required for toxicity by removal of the hypoxic activator (Z₃). In either event, the protected cyclic anthracycline toxins are converted into the corresponding toxic drug in hypoxic tissues by virtue of the activation or reduction of the hypoxic activator moiety (Z₃), resulting in its removal and the concomitant release or generation of the cyclic anthracycline toxin or a modified version of the cyclic anthracycline toxin.

In one embodiment, the trigger is attached to the cyclic anthracycline toxin, in a manner that masks or reduces the cytotoxic activity of the cyclic anthracycline toxin. This masking effect can vary and may depend on the cytotoxic activity of the cyclic anthracycline toxin to be released. Typically, the protected cyclic anthracycline toxin will show at least about 10 fold less cytotoxic activity than the corresponding cyclic anthracycline toxin, and may show up to about a million fold or more or less cytotoxic activity. In one version, the cytotoxic activity of the protected cyclic anthracycline toxin is about 100 fold to about 10,000 fold less than the cytotoxic activity of the corresponding cyclic anthracycline toxin. As one example, for an cyclic anthracycline toxin with an IC₅₀ of 1 nM, the IC₅₀ of the corresponding protected cyclic anthracycline toxin can be 1 microM or greater.

In one version, compounds of Formulas (I) and (II) described herein include as cyclic anthracycline toxin, any agent that can be linked to a hypoxic activator in a manner that yields a protected cyclic anthracycline toxin that is at least about 10-fold to about 1,000,000-fold, and typically about 100 to about 10,000-fold, less active as a cytotoxic agent than the cyclic anthracycline toxin or modified cyclic anthracycline toxin that is released from the compounds of Formulas (I) and (II) under hypoxic conditions.

Possible Mechanism of Action of Protected Cyclic Anthracycline Toxins: Nitroimidazoles have previously been used to form prodrugs for some putative anti-cancer agents, including a PARP inhibitor (see the reference Parveen et al., 1999, Bioorganic and Medicinal Letters 9: 2031-2036) a nitrogen mustard, which was activated, not released, by the nitroimidazole (see the reference Lee et al., 1998, Bioorganic and Medicinal Letters 8: 1741-1744) and the agents described in the A 2-NITROIMIDAZOLE CARBAMATE PRODRUG OF 5-AMINO-1-(CHLOROMETHYL)-3-[5,6,7-TRIMETHOXYINDOL-2-YL)CARBONYL]-1,2-DIHYDRO-3H-BENZ[E]INDOLE (AMINO-SECO-CBI-TMI) FOR USE WITH ADEPT AND GDEPT, M. P. Hay et al., Bioorganic & Medicinal Chemistry Letters 9 (1999) 2237-2242, and PCT publication WO 00/64864. The PARP inhibitor was shown to be released chemically, but no cell culture data was provided. The nitrogen mustard was shown to be active in cell culture data, and the selectivity between normoxic and hypoxic toxicity, while not accurately measured, was stated as greater than 7 fold in cells with normal DNA repair mechanisms.

The compounds of Formulas (I) and (II) described herein differ from such known prodrugs in various ways including but not limited to the nature of the cyclic anthracycline toxin released, the nature of the linking of the hypoxic activator to the cyclic anthracycline toxin the better side effect profile, the presence of more than one hypoxic activator moiety, or some combination of these attributes. Without being bound by theory, these advantages of the protected cyclic anthracycline toxin can be better appreciated with an understanding of the pharmacokinetics of hypoxia-activated prodrugs generally and the protected cyclic anthracycline toxins described herein in particular.

In one version, the protected cyclic anthracycline toxin includes a nitroimidazole as the hypoxic activator. Nitroimidazole is, in the absence of oxygen, converted to a free radical containing moiety by a cytochrome P450 reductase. If the nitroimidazole is appropriately covalently bound to another moiety, further reduction of the free radical form of nitroimidazole can lead to release of that moiety. However, in the presence of oxygen, the free radical reacts with oxygen to form superoxide and the parent nitroimidazole. Superoxide is a cytotoxin, so the production of superoxide in normoxic tissues is believed to lead to unwanted side effects.

Certain nitroimidazole-containing prodrugs can also be activated regardless of the oxygen tension by DT diaphorase, which can lead to activation in normoxic cells, thus contributing to unwanted side effects. Should this normoxic activation pathway create significant side effects with a particular protected cyclic anthracycline toxin, however, one can select another protected cyclic anthracycline toxin that contains more than one hypoxia-activated moiety to reduce or eliminate such side effects. Without being bound by theory, in the case of protected cyclic anthracycline toxins in which the hypoxic activator is a nitroimidazole, the hypoxic activator is activated under hypoxic conditions through the nitro group being reduced to a hydroxylamine or an amine with concomitant release of the portion of the molecule to which the hypoxic activator (Z₃) is attached. This activation process is shown in the following scheme. Without being bound by theory, a mechanism for release from one version of a hypoxic activator (Z₃) is as exemplified in WO 04/87075, which is incorporated herein by reference.

### Methods of Treatment Using the Protected Cyclic Anthracycline Toxins

The compounds of the invention may be used in methods for treating cancer. In such methods, an effective amount of protected cyclic anthracycline toxin is administered to the subject. Generally, the subject may be any human or non-human mammal. The preferred subject is a human subject. Other particular subjects include but are not limited to non-human primates, dogs, cats, farm animals and horses. In one version, the protected cyclic anthracycline toxin is administered alone. In one version the protected cyclic anthracycline toxin is administered in combination with one or more additional anti-cancer agents. In one version the protected cyclic anthracycline toxin is administered in conjunction with a therapeutic cancer treatment, including but not limited to surgery and radiation. The protected cyclic anthracycline toxin will typically be administered in a pharmaceutical composition. Various pharmaceutical compositions that may be used are described in the Formulations section infra.

The protected cyclic anthracycline toxins and their pharmaceutical compositions can be used to treat any type of cancer in a subject, particularly in a human subject. Cancers that may be treated include but are not limited to leukemia, breast cancer, skin cancer, bone cancer, liver cancer, brain cancer, cancer of the larynx, gallbladder, pancreas, rectum, parathyroid, thyroid, adrenal, neural tissue, head and neck, stomach, bronchi, kidneys, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitus tumor, Wilm's tumor, seminoma, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas.

The protected cyclic anthracycline toxins may particularly be used in the treatment of cancers containing significant areas of hypoxic tissue. Such cancers include but are not limited to lung cancer, especially non-small cell lung cancer, breast cancer, colon cancer, head and neck cancer, ovarian cancer, pancreatic cancer, and prostate cancer. Several of these cancers are discussed for illustrative purposes below. Those of skill in the art will appreciate that cancer chemotherapy often involves the simultaneous or successive administration of a variety of anti-cancer agents, and as discussed further below, the protected cyclic anthracycline toxins can be used in combination therapies as provided by the methods described herein. Thus, in the description of illustrative cancers containing hypoxic regions amenable to treatment with the protected cyclic anthracycline toxins, illustrative combination therapies are also described.

Lung cancer affects more than 100,000 males and 50,000 females in the United States, most of whom die within 1 year of diagnosis, making it the leading cause of cancer death. Current protocols for the treatment of lung cancer involve the integration of chemotherapy with or without radiotherapy or surgery. The protected cyclic anthracycline toxins can be used as a single agent or in combination with existing combination therapies. A variety of combination chemotherapy regimens have been reported for small cell lung cancer, including the combinations consisting of cyclophosphamide, doxorubicin and vincristine (CAV); etoposide and cisplatin (VP-16); and cyclophosphamide, doxorubicin and VP-16 (CAVP-16). Modest survival benefits from combination chemotherapy (etoposide plus cisplatin) treatment have been reported for non-small cell lung cancer.

Likewise, several different cytotoxic drugs have produced at least temporary regression of ovarian cancer. The most active drugs in the treatment of ovarian have been alkylating agents, including cyclophosphamide, ifosfamide, melphalan, chlorambucil, thiotepa, cisplatin, and carboplatin. Current combination therapies for ovarian cancer include cisplatin or carboplatin in combination with cyclophosphamide at 3- to 4-week intervals for six to eight cycles. The compounds and methods described herein provide prodrug forms and methods for treating ovarian cancer in which a protected cyclic anthracycline toxin as described herein is used as a single agent or in existing such combination therapy, either to replace an agent or in addition to the agent(s) currently used.

Cancer of the prostate is the most common malignancy in men in the United States and is the second most common cause of cancer death in men above age 55, and this cancer has been reported to consist primarily of hypoxic tissue. Several chemotherapy protocols have been reported for use in late stage disease following relapse after hormonal treatment. Agents for the treatment of prostate cancer include estramustine phosphate, prednimustine, and cisplatin, as well as methods for treating prostate cancer using such agents. Combination chemotherapy is also used to treat prostate cancer, including treatment with estramustine phosphate plus prednimustine and cisplatin, and 5-fluorouracil, melphalan, and hydroxyurea. The compounds and methods described herein provide prodrug forms of cyclic anthracycline toxins, and methods for treating prostate cancer in which a protected cyclic anthracycline toxin is used in such combinations, either to replace an agent or in addition to the agent(s) currently used.

Cancer of the large bowel is the second most common cause of cancer death in the United States and is likewise a cancer characterized by hypoxic regions. While chemotherapy in patients with advanced colorectal cancer has proven to be of only marginal benefit, 5-fluorouracil is the most effective treatment for this disease. 5-Fluorouracil is useful alone or in combination with other drugs, but is associated with only a 15 to 20 percent likelihood of reducing measurable tumor masses by 50 percent or more. Thus, using 5-FU in combination with the compounds and methods described herein, and the methods for treating colon cancer using a prodrug, offer significant therapeutic benefit and potential for meeting the unmet need for better treatment methods for this disease.

In one version of the treatment methods, the protected cyclic anthracycline toxins may be used in various known approaches to cancer therapy including but not limited to “antibody-directed enzyme prodrug therapy” (ADEPT), “virus-directed enzyme prodrug therapy (VDEPT), “gene-directed enzyme prodrug therapy” (GDEPT), and “bacteria-directed enzyme prodrug therapy” (BDEPT). The general uses of the protected cyclic anthracycline toxins are not limited to the foregoing treatment methods.

### Formulations, Modes of Administration, Dosages, etc

The protected cyclic anthracycline toxins will typically be formulated as pharmaceutical formulations for administration to a subject. Described in this section are modes of administration, formulations, and dosages that may be used when treating cancers using the protected cyclic anthracycline toxins described herein.

Administration of the protected cyclic anthracycline toxins for the treatment of cancer can be effected by any method that enables delivery of the prodrugs to the site of action, the hypoxic region of a tumor. Many cancer drugs are administered by intravenous injection, and the protected cyclic anthracycline toxin may be formulated for such administration, including not only ready-for-injection formulations but also lyophilized or concentrated formulations that must be rehydrated or diluted, respectively, prior to injection. In addition to these formulations, the protected cyclic anthracycline toxin may be formulated for administration by oral routes, intraduodenal routes, parenteral injection (including intravenous, subcutaneous, intramuscular, intravascular or infusion), topical, and rectal routes. Those of skill in the art will recognize that the protected cyclic anthracycline toxin can be activated by bacteria in the gut. If such activation is not desired, then the practitioner may employ a route of administration or a formulation that results in absorption of the protected cyclic anthracycline toxin prior to its entry into the large intestine or colon. The actual route of administration and corresponding formulation of the cyclic anthracycline toxins will depend on the type of cancer being treated, the protected cyclic anthracycline toxin selected for administration, the severity of the cancer, and the age, weight, and condition of the patient, among other factors.

In similar fashion, the amount of the protected cyclic anthracycline toxin administered, and thus the amount of the protected cyclic anthracycline toxin contained in the dose administered and the product comprising that dose, will be dependent on the subject being treated, the severity of the cancer, localization of the cancer, the rate of administration, the disposition of the prodrug (e.g., solubility and cytotoxicity), the cytotoxic agent released by the protected cyclic anthracycline toxin, and the discretion of the prescribing physician. However, an effective dosage is typically in the range of about 0.001 to about 100 mg per kg body weight, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect; larger doses can also be divided into several small doses for administration throughout the day.

A formulation of a protected cyclic anthracycline toxin may, for example, be in a form suitable for oral administration as a tablet, capsule, pill powder, sustained release formulation, solution, and suspension; for parenteral injection as a sterile solution, suspension or emulsion; for topical administration as an ointment or cream; and for rectal administration as a suppository. A formulation of a protected cyclic anthracycline toxin may be in unit dosage forms suitable for single administration of precise dosages and will typically include a conventional pharmaceutical carrier or excipient.

Suitable pharmaceutical carriers include inert diluents or fillers, water and various organic solvents. The pharmaceutical compositions may, if desired, contain additional ingredients such as flavorings, binders, excipients, and the like. Thus for oral administration, tablets containing various excipients, such as citric acid may be employed together with various disintegrants, such as starch, alginic acid, and certain complex silicates, and with binding agents such as sucrose, gelatin and acacia. Additionally, lubricating agents such as magnesium stearate, sodium lauryl sulfate, and talc can be used to prepare the tablet forms of formulations of the protected cyclic anthracycline toxins described herein. Solid compositions of a similar type can be employed in soft and hard filled gelatin capsules. Preferred materials, therefore, include lactose or milk sugar and high molecular weight polyethylene glycols. When aqueous suspensions or elixirs are desired for oral administration, the prodrug therein may be combined with various sweetening or flavoring agents, coloring matters or dyes and, if desired, emulsifying agents or suspending agents, together with diluents such as water, ethanol, propylene glycol, glycerin, or combinations thereof.

Exemplary parenteral administration forms include solutions or suspensions of the hypoxia-activated prodrug (protected cyclic anthracycline toxin) in sterile aqueous solutions, for example, aqueous polyethylene glycols, propylene glycol or dextrose solutions. Such dosage forms can be suitably buffered, if desired.

Methods of preparing various pharmaceutical compositions with a specific amount of active drug are known, or will be apparent, to those skilled in this art in view of this disclosure. For examples, see Remington's Pharmaceutical Sciences, Mack Publishing Company, Philadelphia, Pa., 17^{th} Edition (1984).

Combination Therapies

In one version of the method of treating cancer using the protected cyclic anthracycline toxins, a protected cyclic anthracycline toxin is administered in combination with an effective amount of one or more chemotherapeutic agents, an effective amount of radiotherapy, an appropriate surgery procedure, or any combination of such additional therapies.

When a protected cyclic anthracycline toxin is used in combination with one or more of the additional therapies, the protected cyclic anthracycline toxin and additional therapy may be administered at the same time or may be administered separately. For example, if a protected cyclic anthracycline toxin is administered with an additional chemotherapeutic agent, the two agents may be administered simultaneously or may be administered sequentially with some time between administrations. One of skill in the art will understand methods of administering the agents simultaneously and sequentially and possible time periods between administration.

The agents may be administered as the same or different formulations and may be administered via the same or different routes.

Chemotherapeutic agents that may be used in combination with the protected cyclic anthracycline toxins described in this patent include but are not limited to busulfan, improsulfan, piposulfan, benzodepa, carboquone, 2-deoxy-D-glucose, lonidamine and analogs thereof, glufosfamide, meturedepa, uredepa, altretamine, imatinib, triethylenemelamine, triethylenephosphoramide, triethylenethiophosphoramide, trimethylolomelamine, chlorambucil, chlomaphazine, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, carboplatin, defofamide, demecolcine, diaziquone, elformithine, elliptinium acetate, etoglucid, flutamide, gallium nitrate, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, teniposide, tenuazonic acid, triaziquone, 2,2′,2″-trichlorotriethylamine, urethan, vinblastine, cyclophosphamide, and vincristine. Other chemotherapeutic agents that may be used include platinum derivatives, including but not limited to cis platinum, carboplatin, and oxoplatin.

In one version, the protected cyclic anthracycline toxins described herein may be used in combination with an antiangeogenisis inhibitor including but not limited to Avastin and similar therapeutics. In one version of the combination treatment methods, a subject is treated with an antiangeogenisis inhibitor and subsequently treated with a protected cyclic anthracycline toxin. In one version of the combination treatment methods, a subject is treated with an antiangeogenisis inhibitor and subsequently treated with a protected cyclic anthracycline toxin with another chemotherapeutic agent, including but not limited to Cis platinum. In one version of these combination methods of treatment using an antiangeogenisis inhibitor, the method is used to treat breast cancer.

In another version, a protected cyclic anthracycline toxin is administered with an anti-cancer agent that acts, either directly or indirectly, to inhibit hypoxia-inducible factor 1 alpha (HIF1a) or to inhibit a protein or enzyme, such as a glucose transporter or VEGF, whose expression or activity is increased upon increased HIF1a levels. HIF1a inhibitors suitable for use in this version of the methods and compositions described herein include P13 kinase inhibitors; LY294002; rapamycin; histone deacetylase inhibitors such as [(E)-(1S,4S,10S,21R)-7-[(Z)-ethylidene]-4,21-diisopropyl-2-oxa-12,13-dithia-5,8,20,23-tetraazabicyclo-[8,7,6]-tricos-16-ene-3,6,9,19,22-pentanone (FR901228, depsipeptide); heat shock protein 90 (Hsp90) inhibitors such as geldanamycin, 17-allylamino-geldanamycin (17-AAG), and other geldanamycin analogs, and radicicol and radicicol derivatives such as KF58333; genistein; indanone; staurosporin; protein kinase-1 (MEK-1) inhibitors such as PD98059 (2′-amino-3′-methoxyflavone); PX-12 (1-methylpropyl 2-imidazolyl disulfide); pleurotin PX-478; quinoxaline 1,4-dioxides; sodium butyrate (NaB); sodium nitropurruside (SNP) and other NO donors; microtubule inhibitors such as novobiocin, panzem (2-methoxyestradiol or 2-ME2), vincristines, taxanes, epothilones, discodermolide, and derivatives of any of the foregoing; coumarins; barbituric and thiobarbituric acid analogs; camptothecins; and YC-1, a compound described in Biochem. Pharmacol., 15 Apr. 2001, 61(8):947-954, incorporated herein by reference, and its derivatives.

In another version, a protected cyclic anthracycline toxin is administered with an anti-angiogenic agent, including but not limited to anti-angiogenic agents selected from the group consisting of angiostatin, an agent that inhibits or otherwise antagonizes the action of VEGF, batimastat, captopril, cartilage derived inhibitor, genistein, endostatin, interleukin, lavendustin A, medroxypregesterone acetate, recombinant human platelet factor 4, Taxol, tecogalan, thalidomide, thrombospondin, TNP-470, and Avastin. Other useful angiogenesis inhibitors for purposes of the combination therapies provided by the present methods and compositions described herein include Cox-2 inhibitors like celecoxib (Celebrex), diclofenac (Voltaren), etodolac (Lodine), fenoprofen (Nalfon), indomethacin (Indocin), ketoprofen (Orudis, Oruvail), ketoralac (Toradol), oxaprozin (Daypro), nabumetone (Relafen), sulindac (Clinoril), tolmetin (Tolectin), rofecoxib (Vioxx), ibuprofen (Advil), naproxen (Aleve, Naprosyn), aspirin, and acetaminophen (Tylenol). In addition, because pyruvic acid plays an important role in angiogenesis, pyruvate mimics and glycolytic inhibitors like halopyruvates, including bromopyruvate, can be used in combination with an anti-angiogenic compound and a protected cyclic anthracycline toxin to treat cancer. In another version, a protected cyclic anthracycline toxin is administered with an anti-angiogenic agent and another anti-cancer agent, including but not limited to a cytotoxic agent selected from the group consisting of alkylators, Cisplatin, Carboplatin, and inhibitors of microtubule assembly, to treat cancer.

In addition to the combination of a protected cyclic anthracycline toxin with the agents described above, the present methods and compositions described herein provides a variety of synergistic combinations of a protected cyclic anthracycline toxin and other anti-cancer drugs. Those of skill in the art can readily determine the anti-cancer drugs that act “synergistically” with a protected cyclic anthracycline toxin as described herein. For example, the reference Vendetti, “Relevance of Transplantable Animal-Tumor Systems to the Selection of New Agents for Clinical Trial,” Pharmacological Basis of Cancer Chemotherapy, Williams and Wilkins, Baltimore, 1975, and Simpson Herren et al., 1985, “Evaluation of In Vivo Tumor Models for Predicting Clinical Activity for Anticancer Drugs,” Proc. Am. Assoc. Cancer Res. 26: 330, each of which is incorporated herein by reference, describe methods to aid in the determination of whether two drugs act synergistically. While synergy is not required for therapeutic benefit in accordance with the methods of described herein, synergy can improve therapeutic outcome. Two drugs can be said to possess therapeutic synergy if a combination dose regimen of the two drugs produces a significantly better tumor cell kill than the sum of the single agents at optimal or maximum tolerated doses. The “degree of synergy” can be defined as net log of tumor cell kill by the optimum combination regimen minus net log of tumor cell kill by the optimal dose of the most active single agent. Differences in cell kill of greater than ten-fold (one log) are considered conclusively indicative of therapeutic synergy.

When a protected cyclic anthracycline toxin is used with another anti-cancer agent, a protected cyclic anthracycline toxin will, at least in some versions, be administered prior to the initiation of therapy with the other drug or drugs and administration will typically be continued throughout the course of treatment with the other drug or drugs. In some versions, the drug co-administered with a protected cyclic anthracycline toxin will be delivered at a lower dose, and optionally for longer periods, than would be the case in the absence of a protected cyclic anthracycline toxin administration. Such “low dose” therapies can involve, for example, administering an anti-cancer drug, including but not limited to paclitaxel, docetaxel, doxorubicin, cisplatin, or carboplatin, at a lower than approved dose and for a longer period of time together with a protected cyclic anthracycline toxin administered in accordance with the methods described herein. These methods can be used to improve patient outcomes over currently practiced therapies by more effectively killing cancer cells or stopping cancer cell growth as well as diminishing unwanted side effects of the other therapy. In other versions, the other anti-cancer agent or agents will be administered at the same dose levels used when a protected cyclic anthracycline toxin is not co-administered. Thus, when employed in combination with a protected cyclic anthracycline toxin, the additional anti-cancer agent(s) are dosed using either the standard dosages employed for those agents when used without a protected cyclic anthracycline toxin or are less than those standard dosages. The administration of a protected cyclic anthracycline toxin in accordance with the methods described herein can therefore allow the physician to treat cancer with existing (or later approved) drugs at lower doses (than currently used), thus ameliorating some or all of the toxic side effects of such drugs. The exact dosage for a given patient varies from patient to patient, depending on a number of factors including the drug combination employed, the particular disease being treated, and the condition and prior history of the patient, but can be determined using only the skill of the ordinarily skilled artisan in view of the teachings herein.

Specific dose regimens for known and approved chemotherapeutic agents or antineoplastic agents (i.e., the recommended effective dose) are known to physicians and are given, for example, in the product descriptions found in the Physician's Desk Reference 2003, (Physicians' Desk Reference, 57th Ed) Medical Economics Company, Inc., Oradell, N.J. and/or are available from the Federal Drug Administration. Illustrative dosage regimens for certain anti-cancer drugs are also provided below.

Cancer drugs can be classified generally as alkylators, anthracyclines, antibiotics, aromatase inhibitors, bisphosphonates, cyclo-oxygenase inhibitors, estrogen receptor modulators, folate antagonists, inorganic aresenates, microtubule inhibitors, modifiers, nitrosoureas, nucleoside analogs, osteoclast inhibitors, platinum containing compounds, retinoids, topoisomerase 1 inhibitors, topoisomerase 2 inhibitors, and tyrosine kinase inhibitors. In accordance with the methods described herein, a protected cyclic anthracycline toxin can be co-administered with any anti-cancer drug from any of these classes or can be administered prior to or after treatment with any such drug or combination of such drugs. In addition, a protected cyclic anthracycline toxin can be administered in combination with a biologic therapy (e.g., treatment with interferons, interleukins, colony stimulating factors and monoclonal antibodies). Biologics used for treatment of cancer are known in the art and include, for example, trastuzumab (Herceptin), tositumomab and ¹³¹I Tositumomab (Bexxar), rituximab (Rituxan). In one version, however, the anti-cancer drug co-administered with a protected cyclic anthracycline toxin is not a topoisomerase inhibitor.

Alkylators useful in the practice of the methods described herein include but are not limited to busulfan (Myleran, Busulfex), chlorambucil (Leukeran), ifosfamide (with or without MESNA), cyclophosphamide (Cytoxan, Neosar), glufosfamide, melphalan, L-PAM (Alkeran), dacarbazine (DTIC-Dome), and temozolamide (Temodar). In accordance with the methods described herein a protected cyclic anthracycline toxin is co-administered with an alkylator to treat cancer. In one version, the cancer is chronic myelogenous leukemia, multiple myeloma, or anaplastic astrocytoma. As one example, the compound 2-bis[(2-chloroethyl)amino]tetra-hydro-2H-1,3,2-oxazaphosphorine, 2-oxide, also commonly known as cyclophosphamide, is an alkylator used in the treatment of Stages III and IV malignant lymphomas, multiple myeloma, leukemia, mycosis fungoides, neuroblastoma, ovarian adenocarcinoma, retinoblastoma, and carcinoma of the breast. Cyclophosphamide is administered for induction therapy in doses of 1500-1800 mg/m² that are administered intravenously in divided doses over a period of three to five days; for maintenance therapy, 350-550 mg/m² are administered every 7-10 days, or 110-185 mg/m² are administered intravenously twice weekly. In accordance with the methods described herein, a protected cyclic anthracycline toxin is co-administered with cyclosphosphamide administered at such doses or at lower doses and/or for a longer duration than normal for administration of cyclosphosphamide alone.

Anthracyclines useful in the practice of the methods described herein, include but are not limited to doxorubicin (Adriamycin, Doxil, Rubex), mitoxantrone (Novantrone), idarubicin (Idamycin), valrubicin (Valstar), and epirubicin (Ellence). In accordance with the methods described herein a protected cyclic anthracycline toxin is co-administered with an anthracycline to treat cancer. In one version, the cancer is acute nonlymphocytic leukemia, Kaposi's sarcoma, prostate cancer, bladder cancer, metastatic carcinoma of the ovary, and breast cancer. As one example the compound (8S,10S)-10-[(3-Amino-2,3,6-trideoxy-α-L-lyxo-hexopyranosyl)oxy]-8-glycoloyl-7,8,9,10-tetrahydro-6,8,11-trihydroxy-1-methoxy-5,12-naphthacenedione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia, Wilm's tumor, neuroblastoma, soft tissue and bone sarcomas, breast carcinoma, ovarian carcinoma, transitional cell bladder carcinoma, thyroid carcinoma, lymphomas of both Hodgkin and non-Hodgkin types, bronchogenic carcinoma, and gastric carcinoma. Doxorubicin is typically administered in a dose in the range of 30-75 mg/n2 as a single intravenous injection administered at 21-day intervals; weekly intravenous injection at doses of 20 mg/m²; or 30 mg/m² doses on each of three successive days repeated every four weeks. In accordance with the methods of the methods described herein, a protected cyclic anthracycline toxin is co-administered starting prior to and continuing after the administration of doxorubicin at such doses (or at lower doses).

Antibiotics useful in the practice of the methods described herein include but are not limited to dactinomycin, actinomycin D (Cosmegen), bleomycin (Blenoxane), daunorubicin, and daunomycin (Cerubidine, DanuoXome). In accordance with the methods described herein a protected cyclic anthracycline toxin is co-administered with an antibiotic to treat cancer. In one version, the cancer is a cancer selected from the group consisting of acute lymphocytic leukemia, other leukemias, and Kaposi's sarcoma.

Aromatase inhibitors useful in the practice of the methods described herein include but are not limited to anastrozole (Arimidex) and letroazole (Femara). In accordance with the methods described herein a protected cyclic anthracycline toxin is co-administered with an aromatase inhibitor to treat cancer. In one version, the cancer is breast cancer.

Bisphosphonate inhibitors useful in the practice of the methods described herein include but are not limited to zoledronate (Zometa). In accordance with the methods described herein a protected cyclic anthracycline toxin is co-administered with a biphosphonate inhibitor to treat cancer. In one version, the cancer is a cancer selected from the group consisting of multiple myeloma, bone metastases from solid tumors, or prostate cancer.

Cyclo-oxygenase inhibitors useful in the practice of the methods described herein include but are not limited to celecoxib (Celebrex). In accordance with the methods described herein a protected cyclic anthracycline toxin is co-administered with a cyclo-oxygenase inhibitor to treat cancer. In one version, the cancer is colon cancer or a pre-cancerous condition known as familial adenomatous polyposis.

Estrogen receptor modulators useful in the practice of the methods described herein include but are not limited to tamoxifen (Nolvadex) and fulvestrant (Faslodex). In accordance with the methods described herein a protected cyclic anthracycline toxin is co-administered with an estrogen receptor modulator to treat cancer. In one version, the cancer is breast cancer or the treatment is administered to prevent the occurrence or reoccurrence of breast cancer.

Folate antagonists useful in the practice of the methods described herein include but are not limited to methotrexate and tremetrexate. In accordance with the methods described herein a protected cyclic anthracycline toxin is co-administered with a folate antagonist to treat cancer. In one version, the cancer is osteosarcoma. As one example, the compound N-[4-[[(2,4-diamino-6-pteridinyl)methyl methylamino]benzoyl]-L-glutamic acid, commonly known as methotrexate, is an antifolate drug that has been used in the treatment of gestational choriocarcinoma and in the treatment of patients with chorioadenoma destruens and hydatiform mole. It is also useful in the treatment of advanced stages of malignant lymphoma and in the treatment of advanced cases of mycosis fungoides. Methotrexate is administered as follows. For choriocarcinoma, intramuscular injections of doses of 15 to 30 mg are administered daily for a five-day course, such courses repeated as needed with rest period of one or more weeks interposed between courses of therapy. For leukemias, twice weekly intramuscular injections are administered in doses of 30 mg/m². For mycosis fungoides, weekly intramuscular injections of doses of 50 mg or, alternatively, of 25 mg are administered twice weekly. In accordance with the methods described herein, a protected cyclic anthracycline toxin is co-administered with methotrexate administered at such doses (or at lower doses). 5-Methyl-6-[[(3,4,5-trimethoxyphenyl)-amino]methyl]-2,4-quinazolinediamine (commonly known as trimetrexate) is another antifolate drug that can be co-administered with a protected cyclic anthracycline toxin.

Inorganic arsenates useful in the practice of the methods described herein include but are not limited to arsenic trioxide (Trisenox). In accordance with the methods described herein a protected cyclic anthracycline toxin is co-administered with an inorganic arsenate to treat cancer. In one version, the cancer is refractory acute promyelocytic leukemia (APL).

Microtubule inhibitors (as used herein, a “microtubule inhibitor” is any agent that interferes with the assembly or disassembly of microtubules) useful in the practice of the methods described herein include but are not limited to vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. In accordance with the methods described herein a protected cyclic anthracycline toxin is co-administered with a microtubule inhibitor to treat cancer. In one version, the cancer is ovarian cancer, breast cancer, non-small cell lung cancer, Kaposi's sarcoma, and metastatic cancer of breast or ovary origin. As one example, the compound 22-oxo-vincaleukoblastine, also commonly known as vincristine, is an alkaloid obtained from the common periwinkle plant (Vinca rosea, Linn.) and is useful in the treatment of acute leukemia. It has also been shown to be useful in combination with other oncolytic agents in the treatment of Hodgkin's disease, lymphosarcoma, reticulum-cell sarcoma, rhabdomyosarcoma, neuroblastoma, and Wilm's tumor. Vincristine is administered in weekly intravenous doses of 2 mg/m² for children and 1.4 mg/m² for adults. In accordance with the methods described herein, a protected cyclic anthracycline toxin is co-administered with vincristine administered at such doses. In one version, a protected cyclic anthracycline toxin is not administered prior to treatment with a microtubule inhibitor, such as a taxane, but rather, administration of a protected cyclic anthracycline toxin is administered simultaneously with or within a few days to a week after initiation of treatment with a microtubule inhibitor.

Modifiers useful in the practice of the methods described herein include but are not limited to Leucovorin (Wellcovorin), which is used with other drugs such as 5-fluorouracil to treat colorectal cancer. In accordance with the methods described herein a protected cyclic anthracycline toxin is co-administered with a modifier and another anti-cancer agent to treat cancer. In one version, the cancer is colon cancer. In one version, the modifier is a compound that increases the ability of a cell to take up glucose, including but not limited to the compound N-hydroxyurea. N-hydroxyurea has been reported to enhance the ability of a cell to take up 2-deoxyglucose (see the reference Smith et al., 1999, Cancer Letters 141: 85, incorporated herein by reference), and administration of N-hydroxyurea at levels reported to increase a protected cyclic anthracycline toxin uptake or to treat leukemia together with administration of a protected cyclic anthracycline toxin as described herein is one version of the therapeutic methods provided herein. In another such version, a protected cyclic anthracycline toxin is co-administered with nitric oxide or a nitric oxide precursor, such as an organic nitrite or a spermineNONOate, to treat cancer, as the latter compounds stimulate the uptake of glucose and so stimulate the uptake of a protected cyclic anthracycline toxin.

Nitrosoureas useful in the practice of the methods described herein include but are not limited to procarbazine (Matulane), lomustine, CCNU (CeeBU), carmustine (BCNU, BiCNU, Gliadel Wafer), and estramustine (Emcyt). In accordance with the methods described herein a protected cyclic anthracycline toxin is co-administered with a nitrosourea to treat cancer. In one version, the cancer is prostate cancer or glioblastoma, including recurrent glioblastoma multiforme.

Nucleoside analogs useful in the practice of the methods described herein include but are not limited to mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), pentostatin (Nipent), cladribine (Leustatin, 2-CdA), gemcitabine (Gemzar), and capecitabine (Xeloda). In accordance with the methods described herein a protected cyclic anthracycline toxin is co-administered with a nucleoside analog to treat cancer. In one version, the cancer is B-cell lymphocytic leukemia (CLL), hairy cell leukemia, adenocarcinoma of the pancreas, metastatic breast cancer, non-small cell lung cancer, or metastatic colorectal carcinoma. As one example, the compound 5-fluoro-2,4(1H,3H)-pyrimidinedione, also commonly known as 5-fluorouracil, is an antimetabolite nucleoside analog effective in the palliative management of carcinoma of the colon, rectum, breast, stomach, and pancreas in patients who are considered incurable by surgical or other means. 5-Fluorouracil is administered in initial therapy in doses of 12 mg/m² given intravenously once daily for 4 successive days with the daily dose not exceeding 800 mg. If no toxicity is observed at any time during the course of the therapy, 6 mg/kg are given intravenously on the 6th, 8th, 10th, and 12th days. No therapy is given on the 5th, 7th, 9th, or 11th days. In poor risk patients or those who are not in an adequate nutritional state, a daily dose of 6 mg/kg is administered for three days, with the daily dose not exceeding 400 mg. If no toxicity is observed at any time during the treatment, 3 mg/kg may be given on the 5th, 7th, and 9th days. No therapy is given on the 4th, 6th, or 8th days. A sequence of injections on either schedule constitutes a course of therapy. In accordance with the methods described herein, a protected cyclic anthracycline toxin is co-administered with 5-FU administered at such doses or with the prodrug form Xeloda with correspondingly adjusted doses. As another example, the compound 2-amino-1,7-dihydro-6H-purine-6-thione, also commonly known as 6-thioguanine, is a nucleoside analog effective in the therapy of acute non-pymphocytic leukemias. 6-Thioguanine is orally administered in doses of about 2 mg/kg of body weight per day. The total daily dose may be given at one time. If after four weeks of dosage at this level there is no improvement, the dosage may be cautiously increased to 3 mg/kg/day. In accordance with the methods described herein, a protected cyclic anthracycline toxin is co-administered with 6-TG administered at such doses (or at lower doses).

Osteoclast inhibitors useful in the practice of the methods described herein include but are not limited to pamidronate (Aredia). In accordance with the methods described herein a protected cyclic anthracycline toxin is co-administered with an osteoclast inhibitor to treat cancer. In one version, the cancer is osteolytic bone metastases of breast cancer, and one or more additional anti-cancer agents are also co-administered with a protected cyclic anthracycline toxin.

Platinum compounds useful in the practice of the methods described herein include but are not limited to cisplatin (Platinol) and carboplatin (Paraplatin). In accordance with the methods described herein a protected cyclic anthracycline toxin is co-administered with a platinum compound to treat cancer. In one version, the cancer is metastatic testicular cancer, metastatic ovarian cancer, ovarian carcinoma, and transitional cell bladder cancer. As one example, the compound cis-Diaminedichloroplatinum (II), commonly known as cisplatin, is useful in the palliative treatment of metastatic testicular and ovarian tumors, and for the treatment of transitional cell bladder cancer which is not amenable to surgery or radiotherapy. Cisplatin, when used for advanced bladder cancer, is administered in intravenous injections of doses of 50-70 mg/m² once every three to four weeks. In accordance with the methods described herein, a protected cyclic anthracycline toxin is co-administered with cisplatin administered at these doses (or at lower doses). One or more additional anti-cancer agents can be co-administered with the platinum compound and a protected cyclic anthracycline toxin. As one example, Platinol, Blenoxane, and Velbam may be co-administered with a protected cyclic anthracycline toxin. As another example, Platinol and Adriamycin may be co-administered with a protected cyclic anthracycline toxin.

Retinoids useful in the practice of the methods described herein include but are not limited to tretinoin, ATRA (Vesanoid), alitretinoin (Panretin), and bexarotene (Targretin). In accordance with the methods described herein a protected cyclic anthracycline toxin is co-administered with a retinoid to treat cancer. In one version, the cancer is a cancer selected from the group consisting of APL, Kaposi's sarcoma, and T-cell lymphoma.

Topoisomerase 1 inhibitors useful in the practice of the methods described herein include but are not limited to topotecan (Hycamtin) and irinotecan (Camptostar). In accordance with the methods described herein a protected cyclic anthracycline toxin is co-administered with a topoisomerase 1 inhibitor to treat cancer. In one version, the cancer is metastatic carcinoma of the ovary, colon, or rectum, or small cell lung cancer. As noted above, however, in one version of the methods described herein, administration of a protected cyclic anthracycline toxin either precedes or follows, or both, administration of a topoisomerase 1 inhibitor but is not administered concurrently therewith.

Topoisomerase 2 inhibitors useful in the practice of the methods described herein include but are not limited to etoposide, VP-16 (Vepesid), teniposide, VM-26 (Vumon), and etoposide phosphate (Etopophos). In accordance with the methods described herein a protected cyclic anthracycline toxin is co-administered with a topoisomerase 2 inhibitor to treat cancer. In one version, the cancer is a cancer selected from the group consisting of refractory testicular tumors, refractory acute lymphoblastic leukemia (ALL), and small cell lung cancer. As noted above, however, in one version of the methods described herein, administration of a protected cyclic anthracycline toxin either precedes or follows, or both, administration of a topoisomerase 2 inhibitor but is not administered concurrently therewith.

Tyrosine kinase inhibitors useful in the practice of the methods described herein include but are not limited to imatinib (Gleevec). In accordance with the methods described herein a protected cyclic anthracycline toxin is co-administered with a tyrosine kinase inhibitor to treat cancer. In one version, the cancer is CML or a metastatic or unresectable malignant gastrointestinal stromal tumor.

Thus, described herein are methods of treating cancer in which a protected cyclic anthracycline toxin or a pharmaceutically acceptable salt thereof and one or more additional anti-cancer agents are administered to a patient. Specific versions of such other anti-cancer agents include without limitation 5-methyl-6-[[(3,4,5-trimethoxyphenyl)amino]-methyl]-2,4-quinazolinediamine or a pharmaceutically acceptable salt thereof, (8S,10S)-10-(3-amino-2,3,6-trideoxy-alpha-L-lyxo-hexopyranosyl)oxy]-8-glycoloyl-7,8,9,10-tetrahydro-6,8,11-trihydroxy-1-methoxy-5,12-naphthacenedione or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pteridinyl)methyl]-methylamino]benzoyl]-L-glutaric acid, or a pharmaceutically acceptable salt thereof; or cis-diamminedichloroplatinum (II). The methods described herein are generally applicable to all cancers but have particularly significant therapeutic benefit in the treatment of solid tumors, which are characterized by extensive regions of hypoxic tissue. Particular cancers that can be treated with the methods described herein are discussed supra. Methods to synthesize the compounds of the present invention are described in further detail in the “Examples” section below.

### EXAMPLES

In the following examples, any reference to a compound designated by a letter is a reference to the structure shown next to or above that letter in the corresponding reaction schemes.

### Example 1

### Preparation of compound 2a

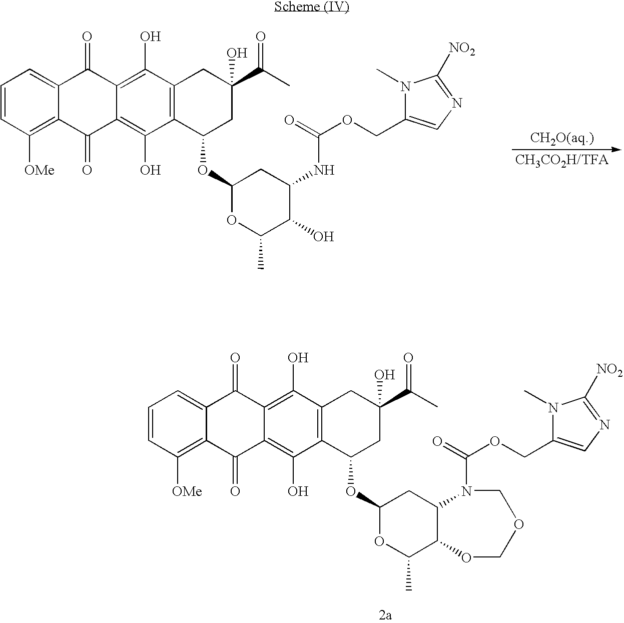

Compound 2a was prepared according to Scheme (IV) as follows.

Into a 50 mL round-bottomed flask was added a mixture of 1-methyl-2-nitroimidazole-5-methanol daunorubicin carbamide (XIII, 100 mg), formaldehyde aqueous solution (40%, 5 mL), acetic acid (5 mL), and TFA (0.1 mL). The flask was wrapped with aluminum foil, and the reaction mixture was stirred at room temperature for 48 hrs. The reaction mixture was extracted with dichloromethane (3×20 mL), and the combined dichloromethane solution was washed with saturated NaHCO₃ aqueous solution (3×20 mL), followed by brine (3×10 mL). After flash column purification (gradient eluent, from AcOEt-Hexane (6:4(v/v)) to AcOEt-MeOH(99:1 (v/v)), pure product was obtained (20 mg). Pure starting material (35 mg) was also recovered from the reaction mixture.

The following compounds 2b and 2c were synthesized using the method followed in Scheme (IV)

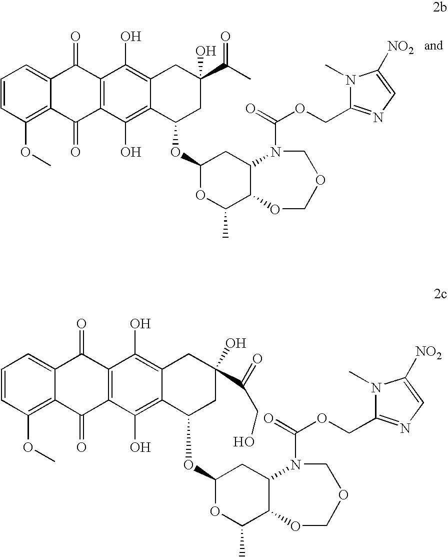

### Example 2

### Preparation of Compound 2d

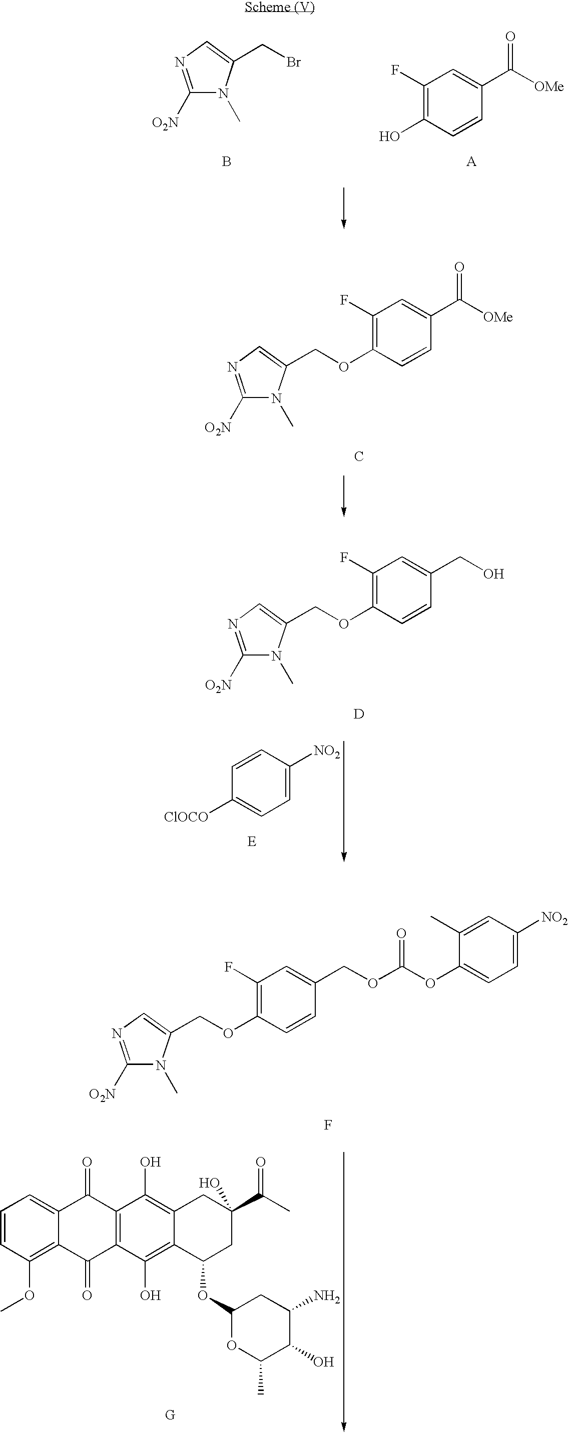

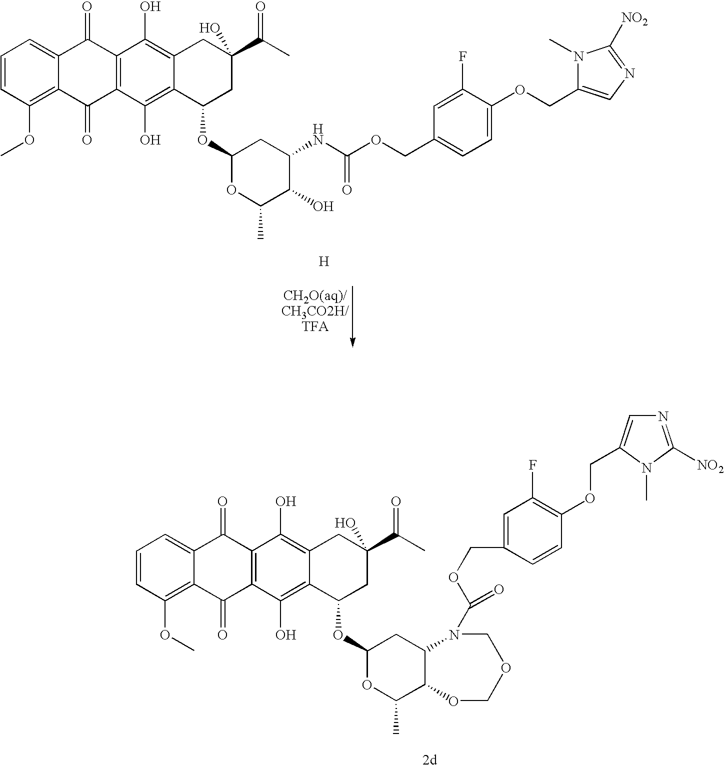

Compound 2d was prepared according to scheme (V) above as follows

Into a 100 mL round-bottomed was added 4-hydroxy-3-fluorobenzoic acid (1 g), methanol (10 mL), and concentrated sulfuric acid (98%, 0.1 mL). The mixture was heated to reflux for 10 hr. After the reaction was completed, the mixture was poured into 100 mL of ice water, and filtered to yield product A (1 g) as a white solid.

Into a 100 mL round-bottomed flask was added a mixture of A (100 mg), B (100 mg), K₂CO₃ (200 mg), and acetone (anhydrous, 1 mL). The mixture was heated to reflux for 4 hr. After the reaction was complete, the reaction mixture was poured into water (10 mL) and extracted with EtOAc (3×15 mL). The combined organic solution was washed with 5% K₂CO₃ (aq., 3×10 mL) to remove excess compound A and dried over Na₂SO₄. The dried organic solution was concentrated to yield compound C (130 mg) as a light yellow solid.

Into a 100 mL round-bottomed flask was added a mixture of C (100 mg), LiBH₄ (2 M in THF, 1 mL), and anhydrous THF (5 mL). The solution was then stirred at room temperature for 24 hr. After the reaction was complete, flash chromatography purification yielded pure alcohol D (60 mg).

Into a 10 mL round-bottomed flask was added a mixture of C (10 mg), THF (2 mL), pyridine (0.1 mL), and p or 4-nitrophenyl chloroformate (E, 10 mg). The mixture was stirred at room temperature for 5 hr. Flash chromatography yielded pure product (F, 14 mg).

Into a 25 mL round-bottomed flask was added a mixture F (8.5 mg), DMF (1 mL), daunorubicin HCl salt (G, 10.7 mg) and DIEA (0.1 mL). The mixture was stirred at room temperature for 2 hr. After the reaction was complete, the mixture was poured into 10 mL dichloromethane and washed with brine (3×5 mL). Flash chromatography gave pure product (H).

Into a 50 mL round-bottomed flask was added a mixture of H (20 mg), formaldehyde aqueous solution (40%, 5 mL), acetic acid (5 mL), and TFA (0.1 mL). The flask was wrapped with aluminum foil, and the reaction mixture was stirred at room temperature for 48 hrs. The reaction mixture was extracted with dichloromethane (3×20 mL), and the combined dichloromethane solution was washed with saturated NaHCO₃ aqueous solution (3×20 mL), followed by brine (3×10 mL). After flash column purification (gradient eluent, from AcOEt-Hexane (6:4(v/v)) to AcOEt-MeOH(99:1(v/v)), pure product was obtained (2d, 5 mg).

In another embodiment, compound 2e, which differs from 2d only in that there is no fluorine attached to the phenyl group in the spacer or fuse between the cytotoxin and the hypoxic activator moiety, is synthesized in accordance with the foregoing protocol.

One of skill in the art will recognizr that compound D in Scheme VI can also be synthesized by first reducing the fluoroester A to the corresponding benzyl alcohol and coupling the benzyl alcohol with compound B as described in Scheme VI.

The following compound (2e) was synthesized using the method followed in Scheme (VI)

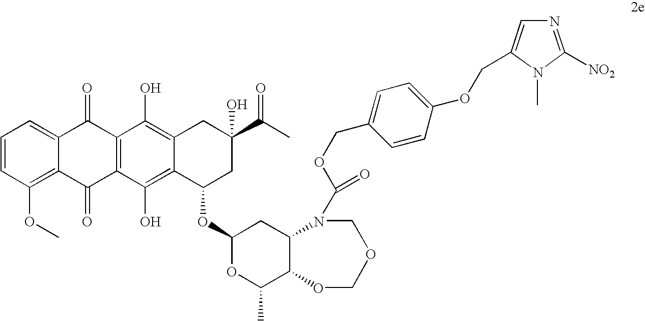

The products synthesized in Examples 1 and 3 were characterized by LC-MS with a major peak of the appropriate molecular weight.

### Example 3

### Preparation of compound 2f

Baminomycin compound 2e is synthesized by reacting Baminomycin (see Perrin et al., supra, incorporated herein by reference) with 4-nitrophenylcarbonate of 1-N-methyl-2-nitroimidazole-5-methanol as shown below.

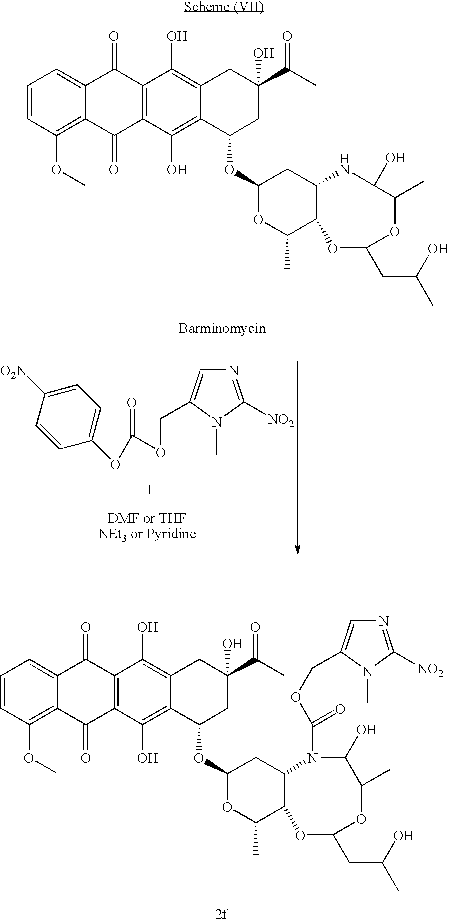

Baminomycin is dissolved in DMF (or THF) at room temperature followed by addition of 4-nitrophenylcarbonate of 1-N-methyl-2-nitroimidazole-5-methanol and triethylamine (or diisopropylamine or pyridine) and the reaction mixture is stirred overnight. Volatiles are removed in vacuo and the solid residue is purified by flash column chromatography on silica gel using dichloromethane-methanol mixture as eluent to separate the desired product. The dichloromethane-methanol mixture which used for TLC of the residue separates (2f) from other products can be used in the flash chromatography.

Baminomycin derivatives:

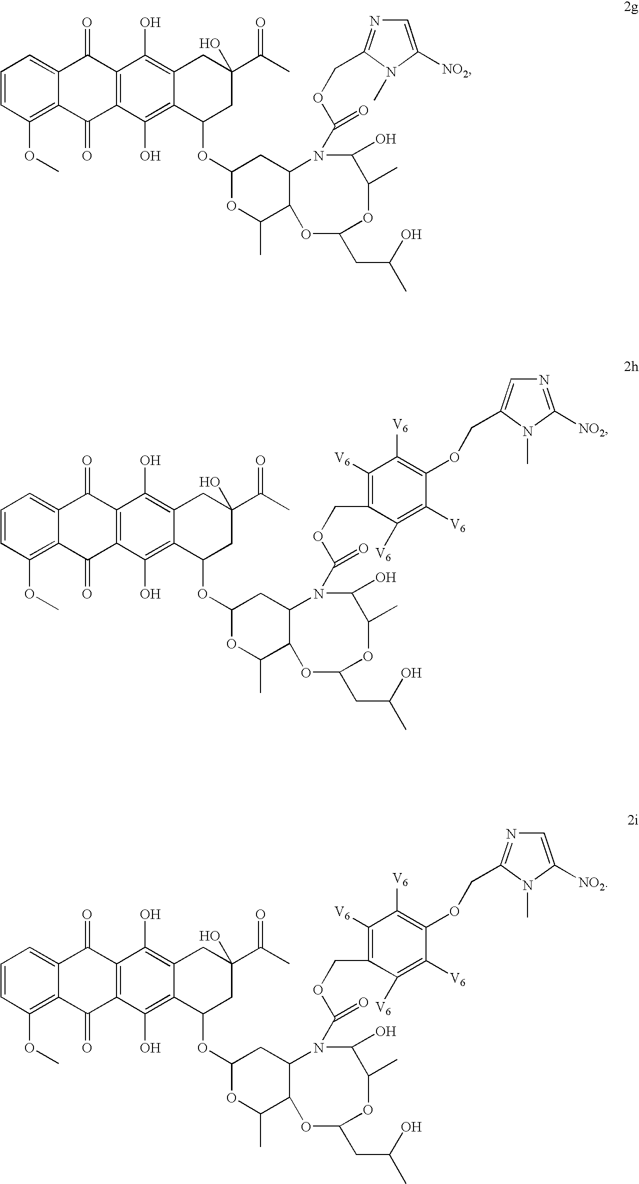

2g-i can be synthesized using the method followed in Schemes (VI) and (VII) while replacing 2-nitroimidazole with 5-nitroimidazole as needed.

Examples 5 and 6 below describe cell-based clonogenic assay for determining cytotoxicity of the compounds of the invention.

### Example 4

Cells are plated in 60-mm glass dishes 1-2 days prior to compound testing at 2×10⁵-1×10⁶ cells per dish. The prodrug to be tested is made up into solution immediately before the test and added to the cells in complete medium. Hypoxia (less than 200 ppm O₂) is achieved by exposing the glass dishes in pre-warmed, air tight aluminum jigs to a series of five rapid evacuations and flushings with 95% nitrogen plus 5% carbon dioxide in a 37 degree C. water bath on a shaking platform (controls are flushed as well). After the fifth evacuation and flushing, the platform (with water bath and jigs) is shaken for 5 minutes, then one more evacuation and flushing are performed, and the jigs are transferred to a shaker in a 37 degree C. incubator for the remainder of the 1-2 hour drug exposure. Levels of oxygenation between 200 ppm and air are achieved by varying the degree and number of evacuations. The oxygen concentrations in the medium and gas phases is checked using an oxygen electrode (Anima, Phoenixville, Pa.) in a specially modified aluminum jig that allows monitoring of both gas and liquid phases. Following the exposure to the drug, the aluminum vessels are opened, the drug washed off the cells by rinsing with medium, the cells trypsinized, and then, the cells are plated for clonogenic survival in plastic Petri dishes. The plating efficiency of the cells should be 60% or greater. Ten to 14 days later, the dishes are stained with crystal violet (0.25% in 95% ethanol), and colonies containing more than 50 cells are counted.

### Example 5

The protected cyclic anthracyclin toxins of the invention (2a-2c) and daunorubicin control were tested in the assay as follows. Exponentially growing human H460 cells (obtained from the ATCC) were seeded into 60 mm notched glass plates at a density of between 2.5 and 5×10⁵ cells per plate and grown in RPMI medium supplemented with 10% fetal bovine serum for 2 days prior to initiating drug treatment. On the day of the test, drug stocks of known concentrations were prepared in complete medium, and 2 ml of the desired stock added to each plate. To achieve complete equilibration between the surrounding gas phase and the liquid phase, the lid of the glass plate was removed and the plate shaken for 5 minutes on an orbital shaker. The plated were recovered and stored inside a glove-box. The glove-box was evacuated and gassed with either a certified anoxic gas mixture (90% nitrogen, 5% hydrogen and 5% carbon dioxide) or with an aerobic (normoxic) gas mixture (95% air and 5% carbon dioxide). Cells were then incubated with the drug for 2 hours at 37° C.

At the end of drug treatment, plates were removed from each vessel, and the drug promptly removed from the cells. Plates were washed with phosphate buffered saline and a solution of trypsin-EDTA and then trypsinized for 5 minutes at 37° C. Detached cells were neutralized with medium plus serum and collected by centrifugation for 5 min at 100×g. Cells were resuspended at approximately 1×10⁶ cells/ml and diluted 10 fold to yield stock concentrations for plating. The concentration of each stock was determined by counting with a Coulter Z2 particle counter. Known numbers of cells were plated, and the plates were placed in an incubator for between 7 and 10 days. Colonies were fixed and stained with a solution of 95% ethanol and 0.25% crystal violet. Colonies of greater than 50 cells were counted, and the surviving fraction was determined.

### Example 6

The protected cyclic anthracyclin toxins of the invention (2d and 2e) and daunorubicin control were tested in the assay as follows. Exponentially growing human H460 cells (obtained from ATCC) were seeded into 60 mm notched glass plates between 2.5 and 5×10⁵ cells per plate and grown in RPMI medium supplemented with 10% fetal bovine serum for 2 days prior to initiating treatment. On the day of the experiment drug stocks of known concentrations were prepared in complete medium and 2 ml added to each plate. Glass plates were sealed into airtight aluminum vessels equipped with a valve to control gas flow. To achieve complete equilibration between the gas phase and the liquid phase a series of gas exchanges were performed on each vessel while shaking. Vessels were evacuated and gassed with either a certified anoxic gas mixture (95% nitrogen and 5% carbon dioxide) or with aerobic gas mixture (95% air and 5% carbon dioxide). Specifically, each vessel was evacuated to minus 26 inches of mercury and held for 15 seconds before gassing at 20 psi and again holding for 15 seconds. After a series of five evacuations and gassings the vessels were held an additional 5 minutes before a final evacuation and refilling of each vessel with the desired gas mixture at 0.5 psi above atmospheric pressure. Cells were incubated for 2 hours at 37° C. At the end of treatment, plates were removed from each vessel and the drug promptly removed from the cells. Plates were washed with phosphate buffered saline and a solution of trypsin-EDTA and then trypsinized for 5 minutes at 37° C. Detached cells were neutralized with medium plus serum and spun for 5 min at 100×g. Cells were resuspended at approximately 1×10⁶ cells/ml and diluted 10 fold to yield stock concentrations for plating. The exact concentration of each stock was determined by counting with a Coulter Z2 particle counter. Known numbers of cells were plated and placed undisturbed in an incubator for between 7 and 10 days. Colonies were fixed and stained with a solution of 95% ethanol with 0.25% crystal violet stain. Colonies of greater than 50 cells were counted and the surviving fraction determined.

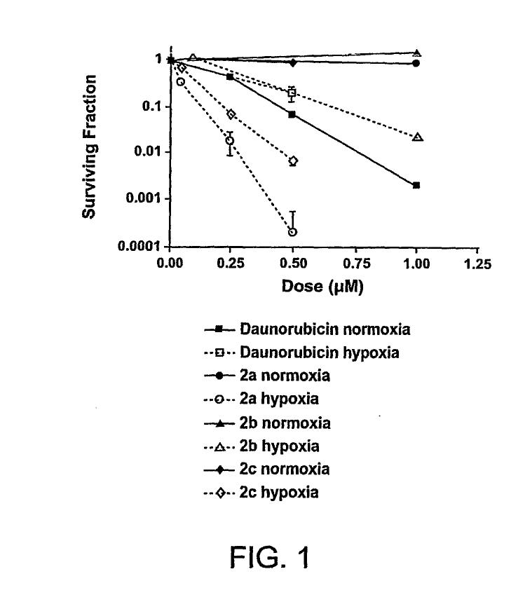

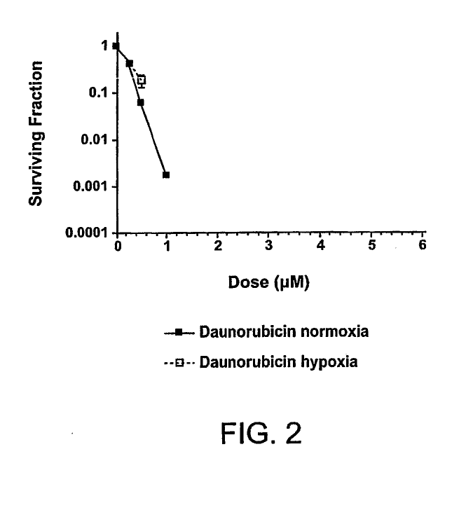

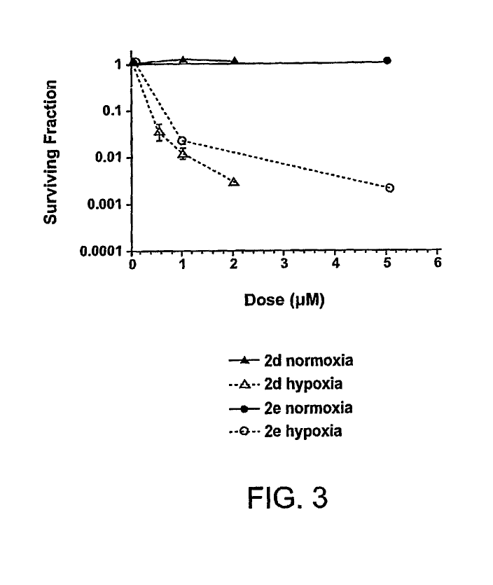

The results from the clonogenic assays performed as described above (Example 6) and in Example 5 are summarized in FIGS. 1-3. FIGS. 1-3 illustrate the dose response profile for compounds of the invention (2a-e) as compared to daunorubicin under normoxic conditions (normoxia) and hypoxic conditions (hypoxia) as determined by fraction of surviving cells. As illustrated in the figures, compounds 2a-e were less toxic than daunorubicin under normoxia and more or about as toxic as daunorubicin under hypoxia.

Compounds of the invention were also tested in HT 29 and SCC VII cell based clonogenic assays in the same way as described above in Example 5 and this example.

### Example 7

CD1 Mice were injected intravenously with a single 20 mg/kg dose each of compounds 2a, 2b, daunorubicin, and vehicle and observed for seven days, before euthanization. Significant weight loss was observed in mice injected with Daunorubicin. Mice injected with 2a or 2b did not undergo significant weight loss as compared with the control group. Thus, compounds of the invention appear to be less toxic than Daunorubicin when administered to healthy mice.

All publications and patent documents cited herein are incorporated herein by reference as if each such publication or document was specifically and individually indicated to be incorporated herein by reference.

We claim:

1. A compound having formula: 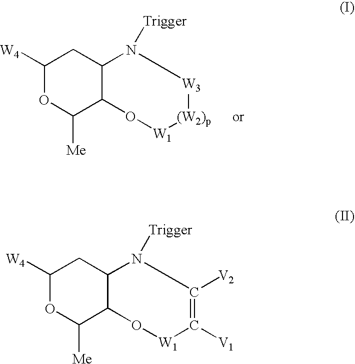
wherein
p is 1 or 2;
W₁ is C(V₁)₂, C═O, or SO₂;
W₂ is C(V₁)₂, NV₁, O, or S with the proviso that when p is 2 both W₂ are not O;
W₃ is CV₁V₂ wherein each V₁ is independently hydrogen, C₁-C₆ alkyl or heteroalkyl and
V₂ is hydrogen, hydroxy, mercapto, C₁-C₆ alkylthio or C₁-C₆ alkoxy;
W₄ is: 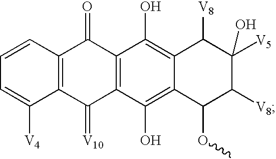
wherein V₄ is hydrogen, C₁-C₆ alkyl or heteroalkyl, hydroxy, C₁-C₆ alkoxy, amino, C₁-C₆ alkylamino, C₁-C₆ dialkylamino, mercapto, and C₁-C₆ alkylthio; V₅ is selected from the group consisting of —CH₂CH₃, —COCH₃, —CH(OH)CH₃, —COCH₂OH, —CH(OH)CH₂OH, —C(═N-Z₁)-CH₃, and —C(═N-Z₁)-CH₂OH wherein Z₁ is —OZ₂ or —N(Z₂)₂ wherein each Z₂ is selected from the group consisting of hydrogen, C₁-C₆-acyl or heteroacyl, aroyl or heteroaroyl, C₁-C₆ alkyl or heteroalkyl, and aryl or heteroaryl;
V₁₀ is O or NH;
each V₈ is halo or hydrogen provided that they are both not halo
Trigger is —[C(Z₄)₂-Z₇]_{w}—(C(═O)—O)_{q}—[C(Z₄)₂-Z₅-Z₆]ᵤ—C(Z₄)₂[—C(Z₄)═C(Z₄)]₁-Z₃ or —[C(Z₄)₂-Z₇]_{w}—(S(═O)₂)_{q}—[C(Z₄)₂-Z₅-Z₆]ᵤ—C(Z₄)₂-[C(Z₄)═C(Z₄)]₁-Z₃,
wherein each w, q, u, and independently is 0 or 1; each Z₄ independently is hydrogen, halo, C₁-C₆ alkyl or heteroalkyl, aryl or heteroaryl, C₁-C₆ acyl or heteroacyl, aroyl, or heteroaroyl;
Z₃ is selected from the group consisting of: 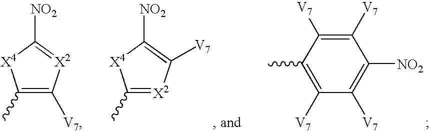
wherein X⁴ is NV¹, O, or S wherein V¹ is defined as before; each X² is N or CV⁷ wherein each V⁷ is C₁-C₆ alkyl or heteroalkyl, aryl, hydrogen, halogen, nitro, C₁-C₆ alkoxy, cyano, CO₂H, or CON(V₁)₂. 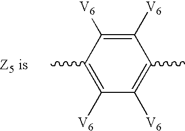
wherein each V₆ is hydrogen, halo, nitro, C₁-C₆ alkoxy, cyano, CO₂H, CON(V₁)₂;
Z₆ is S, O, or NV₁—(C(═O)—O)ᵥ wherein V₁ is defined as above and v is 0 or 1 provided that if v is 0, then Z₆ excludes NH;
Z₇ is S, O, or NV₁ provided that if Z₇ is S or O then q=0; and
an individual isomer or a racemic or non-racemic mixture of isomers, a pharmaceutically acceptable salt, solvate, hydrate, or a prodrug thereof.

2. The compound of claim 1 wherein W₄ is: 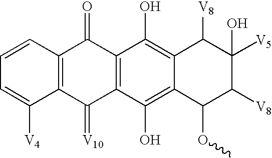
wherein
V₄ is hydrogen, methoxy, or hydroxy;
V₅ is —CH₂CH₃, —COCH₃, —CH(OH)CH₃, —COCH₂OH, —CH(OH)CH₂OH, —C(═N—NHCOPh)—CH₃, or —C(═N—NHCOPh)—CH₂OH;
V₁₀ is O or NH; and
V₈ is hydrogen or fluoro; and
an individual isomer or a racemic or non-racemic mixture of isomers, a pharmaceutically acceptable salt, solvate, hydrate, or a prodrug thereof.

3. The compound of claim 2 wherein Z₃ is selected from the group consisting of: 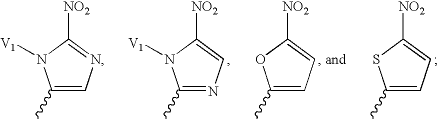
wherein V₁ is C₁-C₆ alkyl or heteroalkyl; and
an individual isomer or a racemic or non-racemic mixture of isomers, a pharmaceutically acceptable salt, solvate, hydrate, or a prodrug thereof.

4. The compound of claim 2 wherein -Z₅-Z₆- together is selected from the group consisting of: 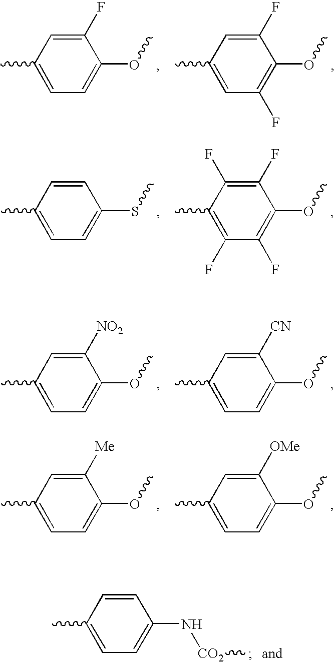
an individual isomer or a racemic or non-racemic mixture of isomers, a pharmaceutically acceptable salt, solvate, hydrate, or a prodrug thereof.

5. The compound of claim 2 of formula: 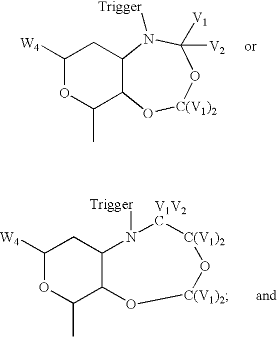
an individual isomer or a racemic or non-racemic mixture of isomers, a pharmaceutically acceptable salt, solvate, hydrate, or a prodrug thereof.

6. The compound of claim 2 having the formula: 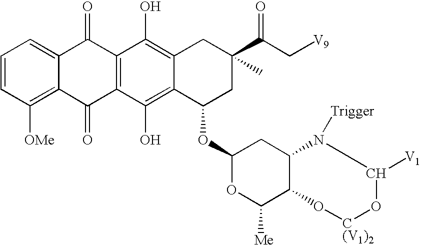
wherein each V₁ is C₁-C₆ alkyl and heteroalkyl and V₉ is H or OH; and an individual isomer or a racemic or non-racemic mixture of isomers, a pharmaceutically acceptable salt, solvate, hydrate, or a prodrug thereof.

7. The compound of claim 2 having the formula: 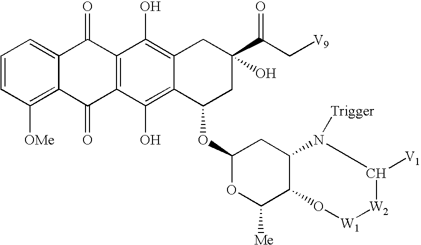
wherein W₁ is CO or SO₂; W₂ is NV₁ wherein each V₁ is C₁-C₆ alkyl or heteroalkyl; and V₉ is H or OH; and
an individual isomer or a racemic or non-racemic mixture of isomers, a pharmaceutically acceptable salt, solvate, hydrate, or a prodrug thereof.

8. The compound of claim 2 having the formula: 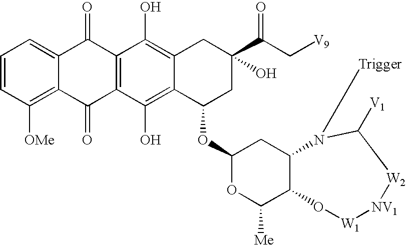
wherein W₁ is CO or SO₂; W₂ is NV₁ or O wherein each V₁ is C₁-C₆ alkyl; and V₉ is H or OH; and
an individual isomer or a racemic or non-racemic mixture of isomers, a pharmaceutically acceptable salt, solvate, hydrate, or a prodrug thereof.

9. The compound of claim 2 having the formula: 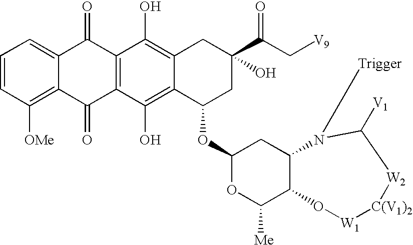
wherein W₁ is CO or SO₂; W₂ is NV₁, O, or S wherein each V₁ is hydrogen, C₁-C₆ alkyl, C₁-C₆ alkoxy, C₁-C₆ thioalkyl, hydroxy, or mercapto; and V₉ is H or OH; and
an individual isomer or a racemic or non-racemic mixture of isomers, a pharmaceutically acceptable salt, solvate, hydrate, or a prodrug thereof.

10. The compound of claim 2 having the formula: 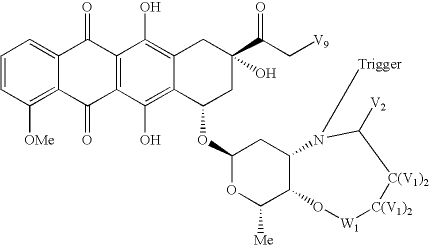
wherein W₁ is CO or SO₂ and V₉ is H or OH; and
an individual isomer or a racemic or non-racemic mixture of isomers, a pharmaceutically acceptable salt, solvate, hydrate, or a prodrug thereof.

11. In a related embodiment, the present invention provides a compound of formula: 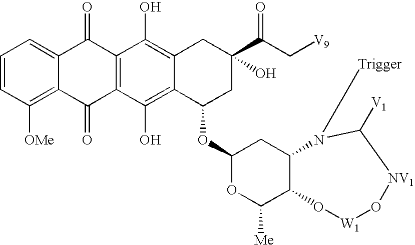
wherein W₁ is CO or SO₂ and V₉ is H or OH; and
an individual isomer or a racemic or non-racemic mixture of isomers, a pharmaceutically acceptable salt, solvate, hydrate, or a prodrug thereof.

12. The compound of claim 2 having the formula: 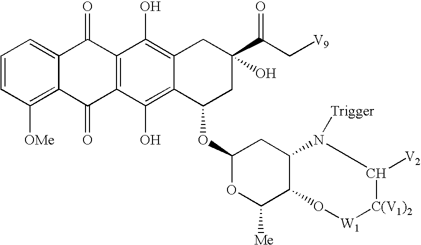
wherein W₁ is CO or SO₂ and V₉ is H or OH; and
an individual isomer or a racemic or non-racemic mixture of isomers, a pharmaceutically acceptable salt, solvate, hydrate, or a prodrug thereof.

13. In another embodiment, the present invention provides a compound of formula 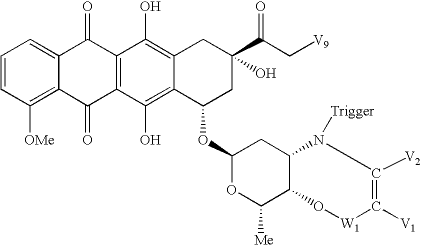
wherein W₁ is CO or SO₂ and V₉ is H or OH; and
an individual isomer or a racemic or non-racemic mixture of isomers, a pharmaceutically acceptable salt, solvate, hydrate, or a prodrug thereof.

14. The compound of claim 5 of formula: 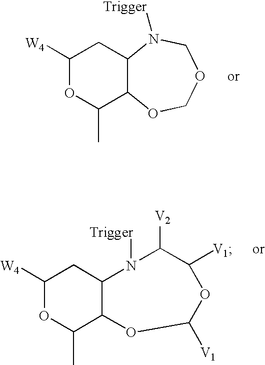
an individual isomer or a racemic or non-racemic mixture of isomers, a pharmaceutically acceptable salt, solvate, hydrate, or a prodrug thereof.

15. The compound of claim selected from the group consisting of: 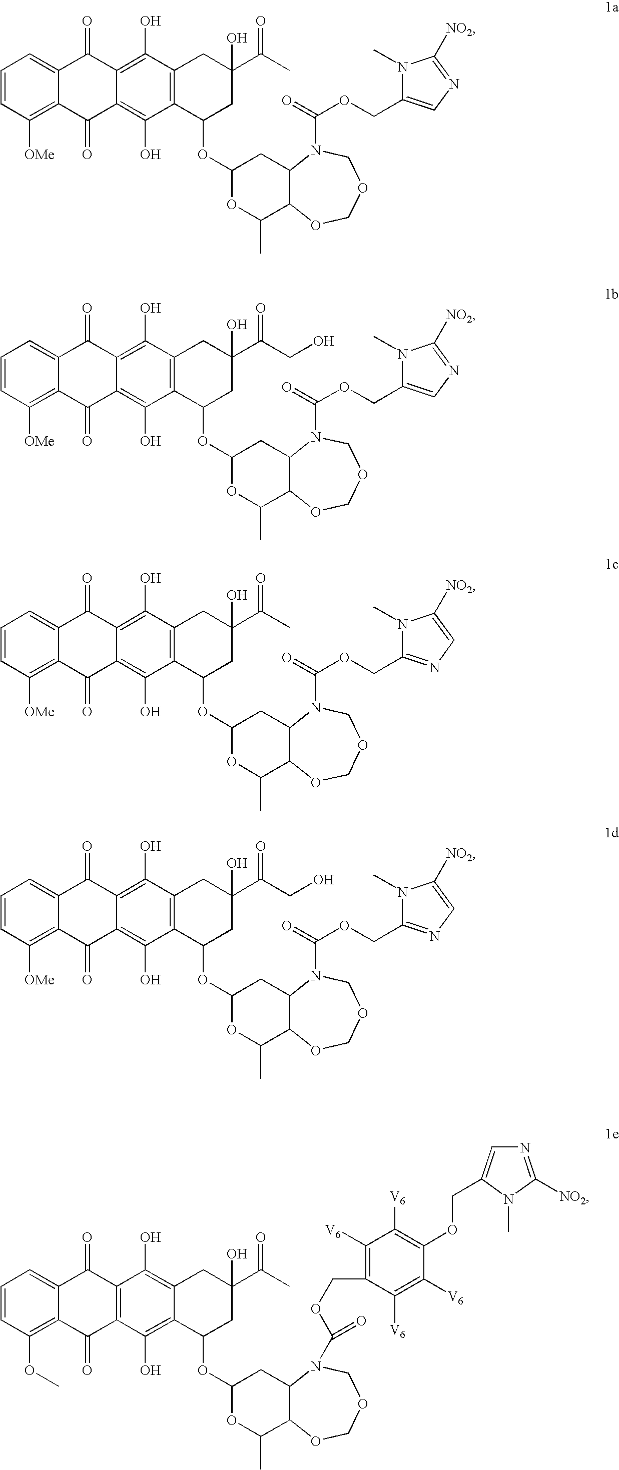 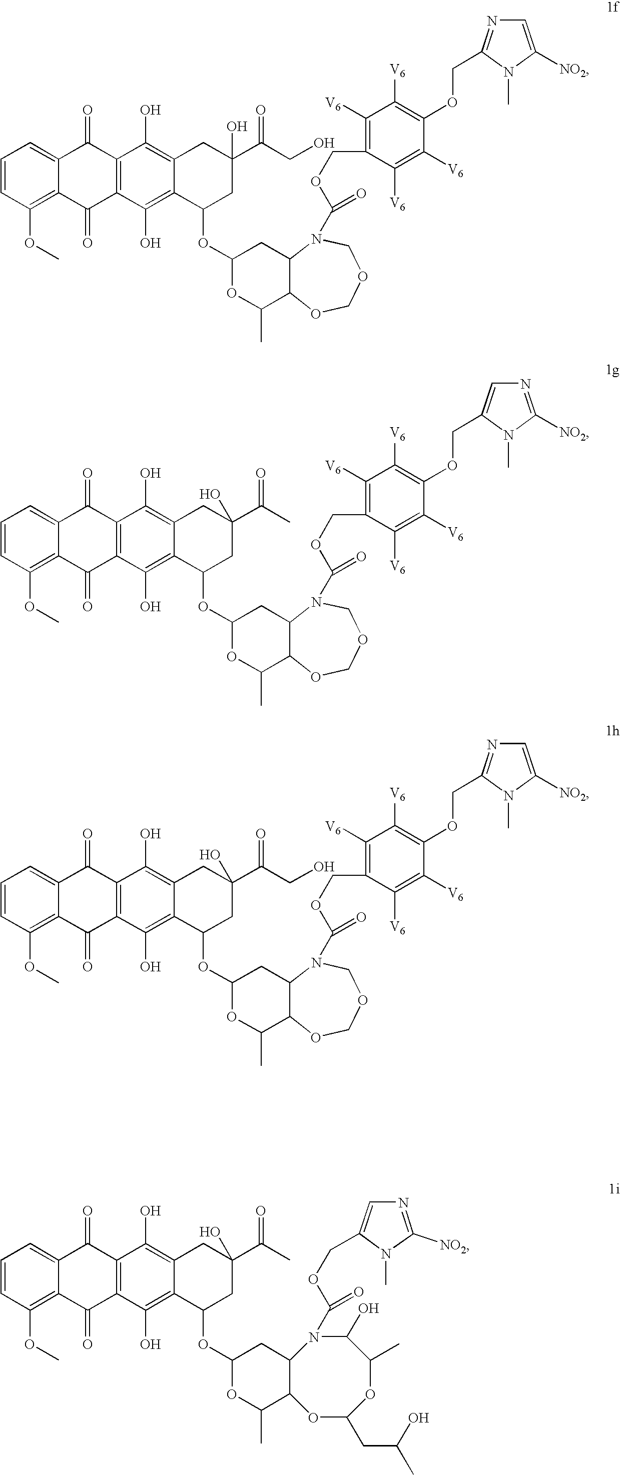 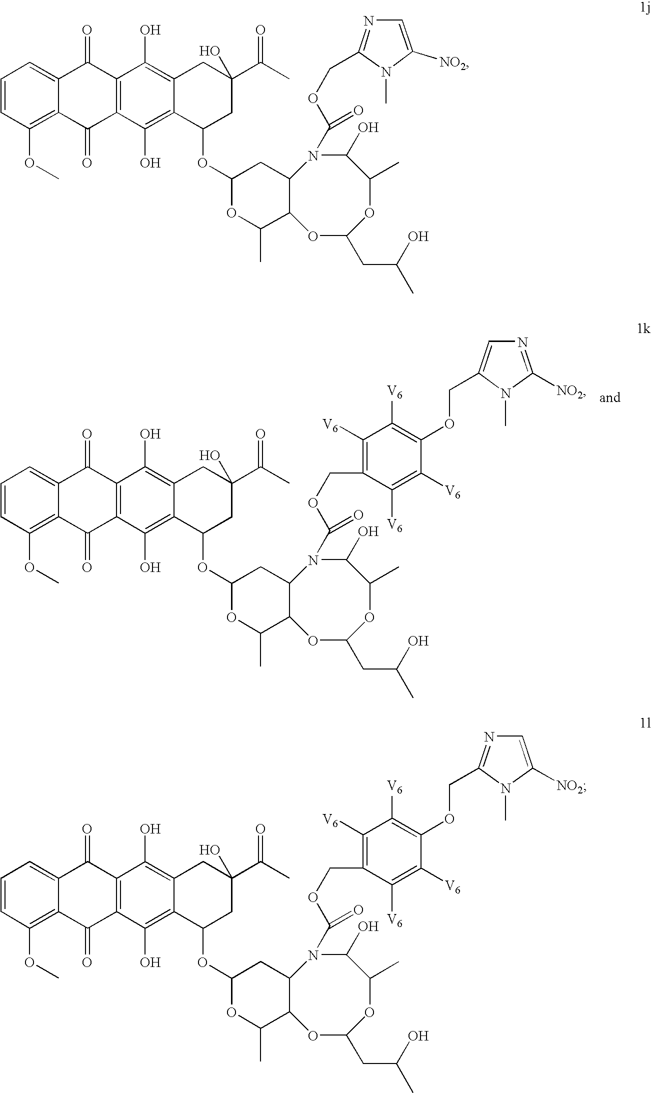
wherein each V₆ independently is fluoro or hydrogen; and
an individual isomer or a racemic or non-racemic mixture of isomers, a pharmaceutically acceptable salt, solvate, hydrate, or a prodrug thereof.

16. A pharmaceutical composition comprising a compound of any one of claims 1-15 and a pharmaceutical carrier or excipient.

17. A method of treating cancer in a patient in need of therapy thereof, said method comprising administering a therapeutically effective dose of the pharmaceutical composition of claim 16.

18. The method of claim 17 further comprising administering a therapeutically effective amount of one or more chemotherapeutic agents, an effective amount of radiotherapy, a surgery procedure, or any combination of the foregoing.

19. The method of claim 18, wherein said chemotherapeutic agent is cytotoxic or cytostatic against normoxic cells.
